(19) United States
(12) Patent Application Publication
(10) Pub. No.: US 2023/0058171 A1
(43) Pub. Date: Feb. 23, 2023

(54) NEW ORAL PHARMACEUTICAL COMPOSITION FOR CANCER THERAPY

(71) Applicant: BOEHRINGER INGELHEIM INTERNATIONAL GMBH, Ingelheim am Rhein (DE)

(72) Inventors: Mehdi Mourad LAHMAR, Mainz (DE); Junxian GENG, Danbury, CT (US); Rolf GREMPLER, Mittelbiberach (DE); Alejandro PEREZ-PITARCH, Ingelheim am Rhein (DE); Maren ROHRBACHER, Mannheim (DE)

(21) Appl. No.: 17/818,009

(22) Filed: Aug. 8, 2022

(30) Foreign Application Priority Data
Aug. 9, 2021 (EP) 21190294.5
Feb. 10, 2022 (EP) 22156077.4
May 25, 2022 (EP) 22175571.3

(51) Int. Cl. A61K 31/4162; A61K 9/00; A61K 39/395; A61P 35/00

(52) U.S. Cl. CPC A61K 31/4162; A61K 9/0053; A61K 39/3955; A61P 35/00

(57) ABSTRACT

One aspect of the invention refers to an oral pharmaceutical composition comprising the MDM2-antagonist of formula I

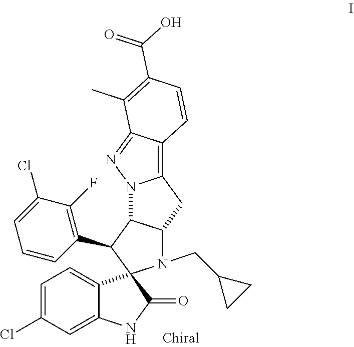

in a dose range of 30 mg to 45 mg for use in the treatment of cancer, wherein this oral pharmaceutical composition is administered in a treatment cycle of once every three weeks (D1 q3w), wherein this treatment cycle of once every three weeks (D1 q3w) may be repeated that many times as considered beneficial for the patient from a medical point of view.

Another aspect of the invention refers to an MDM2-antagonist of formula I for use in the first line systemic treatment (primary treatment) of dedifferentiated liposarcoma.

A further aspect of the invention refers to the use of the MDM2-antagonist of formula I for the manufacture of a medicament for the treatment of a p53-wildtype and non-MDM2-amplified form of cancer.

### 1. BACKGROUND

### 1.1 Sarcomas

Sarcomas are a rare and heterogeneous group of malignant tumours of mesenchymal origin that comprise <1% of all adult malignancies and 12% of paediatric cancers. Approximately 80% of sarcomas originate from soft tissue, and the rest originate from bone. More than 100 histologically and biologically distinct subtypes of sarcoma are recognized with the majority being Soft Tissue Sarcomas (STS). The most common STS subtypes in adults are undifferentiated/unclassified STS, undifferentiated pleiomorphic sarcoma (UPS), leiomyosarcoma (LMS), gastrointestinal stromal tumours (GIST) and liposarcoma (LPS). Some 13,000 new patients with STS are reported each year in the US (Gamboa et al, CA Cancer J Clin 2020; 70:200-229; Corey et al, Cancer Medicine 2014, Vol. 3 (5), 1404-1415; Bock et al, Int. J. Environ. Res. Public Health 2020, 17, page 2710 ff; Amer et al, J. Clin. Orthopaedics and Trauma 11 (2020) S479-S484). The RARECARE project involving 76 population-based cancer registries reported an estimated 23,574 new cases of STS in the EU each year, with age-standardized incidence rates per 100,000 ranging from 3.3 in Eastern Europe to 4.7 in Northern Europe (Stiller et al, European J. Canc. (2013), Vol. 49, pp. 684-695). A recent report by Orphanet in January 2021 estimated the prevalence and incidence of STS in Europe to be 30.0 per 100,000 and 4.74 per 100,000, respectively (Orphanet Report Series, Prevalence of rare diseases, No. 1, January 2021).

While surgical management remains the mainstay of the treatment of localized STS, several different chemotherapy regimens are used in advanced/metastatic STS. First line systemic therapy has not changed substantially for the last 45 years and is still based on doxorubicin-containing chemotherapy. The latest trials enrolling patients with STS showed a median progression free survival (mPFS) of 7 months and an overall survival (OS) rate of around 18 months. However, since patients' prognosis varies from one histology to another, PFS and OS can be quite different depending on the specific STS histology (Gino et al, Ther Adv Med Oncol. 2017, Vol. 9 (8), pp 533-550; Savina et al, BMC Medicine (2017), 15, pp. 78; Tap et al, JAMA, 2020; Vol. 323 (13): pp. 1266-1276).

LPS represents 20% of STS and includes four different subtypes: Well differentiated and dedifferentiated (over 90% of these tumours are MDM2 amplified) as well as round cell/myxoid & pleiomorphic subtypes (lower prevalence of MDM2 amplification <10%).

The dedifferentiated LPS subtype (DDLPS) represents 15-20% of all LPS patients and represents a high-grade tumour that metastasizes in more than 20% of cases (lungs, liver, bone, skin or brain) and with low responsiveness to doxorubicin; the following outcomes after 1st line therapy have been reported in small retrospective studies and outline the high unmet medical need in this niche indication: ORR<15%/mPFS: 2-4 mo/mOS: 8-12 mo. (Italiano et al, Ann. Oncol. (2012), Vol. 23, No. 6, pp. 1601-1607; Savina et al, BMC Medicine (2017), Vol. 15, pp. 78; Langsman et al, Oncol Res Treat, 2019, Vol. 42, pp. 396-403; Gahvari et al, Curr. Treat. Options in Oncol. (2020), Vol. 21, pp. 15).

Patients suffering from STS report the following symptoms which strongly impacts their daily functioning: pain (specifically related to the tumour location, i.e., abdominal pain, muscle pain, bone pain), fatigue, dyspnoea, as well as eating and sleeping problems. These symptoms increase with disease progression, limiting their daily activities and causing negative emotions, worsening of their disease-related distress. The impact of these symptoms is comparable to patients with other metastatic cancers. The symptomatic experience of patients with LPS are like that of other STS patients, although pain experience is dependent on the tumour location (Winnette et al, Patient (2017), Vol. 10, pp. 153-162; den Hollander et al, ESMO Open Cancer Horizons 2020, 5: e000914).

### 1.2 TP53 and MDM2

The protein TP53 (p53), the so-called “guardian of the genome”, is a pivotal tumor suppressor protein and a mainstay of the body's cellular anti-cancer defense system (Lane et al, Nature (1992); Vol. 358 (6381): 15-16). As a transcription factor, p53 regulates multiple downstream target genes that are involved in cell cycle arrest or senescence, DNA repair and apoptosis (Donehower et al, Nature (1992); 356 (6366): 215-221; Olivier et al, Cold Sping Harbor Perspectives in Biology (2010); 2 (1): a001008-a001008; Levine et al, Nature Reviews Cancer 2009, 9 (10): 749-758). Under normal conditions it is therefore critical that intracellular levels of p53 are kept at a low, basal state, which is achieved by rapid (proteasome-mediated) degradation of p53 (Brooks et al Molecular Cell 2006; 21 (3): 307-315). In cells that are exposed to stress signals or are damaged, TP53 is rapidly activated, while it is kept in check in normal cells that are not exposed to stress signals and in tumor cells, in which the TP53 gene is frequently mutated. Although the incidence of TP53 mutations differs significantly between different cancer types, TP53 is one of the most frequently mutated genes in human cancers with about 50% of all cancers having mutations or deletions in this gene (Kandoth et al, Nature, 2013, 502 (7471): 333-339; Lawrence et al, Nature, 2014, 505 (7484): 495-501). The remaining human cancers have tumors with TP53 wild-type status but the function of p53 is frequently attenuated by other mechanisms, including overexpression or amplification of its key negative regulator, human MDM2.

MDM2 antagonists block the interaction between p53 and its key negative regulator, MDM2, and represent a new therapeutic concept for cancer. MDM2 antagonist are designed to restore p53 activity in TP53 wild-type tumors and several MDM2 antagonists are currently being evaluated for clinical development.

The analyses described in Oliner et al, Cold Spring Harb Perspect Med 2015; 6: a026336 implicate that MDM2 amplification is the dominant mechanism through which human tumors raise MDM2 levels to abrogate p53. Therefore it is not surprising that small molecule antagonists of MDM2 are expected to have the best therapeutic capacities in tumors that are p53 wildtype and that are additionally MDM2-amplified, such as e.g. liposarcomas, in particular well-differentiated and dedifferentiated liposarcomas.

### 1.3 MDM2-Antagonist of Formula I

The MDM2-p53 antagonist of formula I

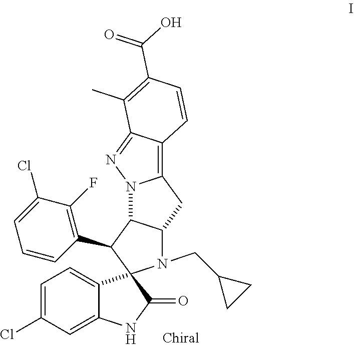

((2′S,3′S,3a'S,10a'S)-6-chloro-3′-(3-chloro-2-fluorophenyl)-1′-(cyclopropylmethyl)-6′-methyl-2-oxo-1,2,3′,3a′,10′,10a′-hexahydro-1′H-spiro[indole-3,2′-pyrrolo[2′,3′:4,5]pyrrolo[1,2-b]indazole]-7′-carboxylic acid) is a new small molecule that inhibits the interaction between the tumour suppressor protein p53 (TP53) and its negative regulator murine double minute 2 (MDM2) (see WO 2017/060431, example Ia-34). MDM2 functions as an effective TP53 antagonist in cells with WT TP53. In human tumours, overexpression of the MDM2 protein can be caused by gene amplification. The MDM2 gene is amplified in 7% of human cancers across tumour types. Gene amplification of MDM2 occurs at high frequency in LPS (>50%) and even more so in well-differentiated and dedifferentiated LPSs (>90%). MDM2 amplifications also occur at lower frequency in lung adenocarcinoma (4.6%), urothelial carcinoma (9%) and glioblastoma multiforme (9%).

Inhibition of the interaction between TP53 and MDM2 leads to stabilization of TP53 followed by target gene induction, which results in cell cycle arrest or apoptosis in tumour cells with TP53 wild-type status. Non-clinical data suggest that the anti-tumour efficacy of the MDM2 antagonist of formula I in tumours harbouring wild-type TP53 is based on two modes of action (MoA): direct anti-tumour activity via activation of wild-type TP53 function and induction of apoptosis in cancer cells (MoA1), and immunomodulatory activity, with synergistic efficacy in combination with immune checkpoint antagonists such as e.g. immune checkpoint antagonists targeting PD-1 (MoA2).

### 1.4 Ezabenlimab and Other PD-1 Axis Inhibitors

Ezabenlimab is a mouse derived, monoclonal IgG4Pro antibody (mAb) targeted to the human programmed cell death-1 (PD-1) immune checkpoint antagonist. Binding of the PD-1 ligands, PD-L1 and PD-L2, to the PD-1 receptor found on T cells, inhibits T cell proliferation and cytokine production. Upregulation of PD-1 ligands occurs in some tumours and signalling through this pathway can contribute to inhibition of active T cell immune surveillance of tumours. Ezabenlimab is a mouse derived, humanized monoclonal IgG4Pro mAb targeted to the human (PD-1) protein and potently blocks PD-1/PD-L1 and PD-1/PD-L2 interactions in vitro.

By combining the MDM2-antagonist of formula I and ezabenlimab, synergistic efficacy via immunomodulatory activity is proposed and induction of anti-tumour immunity based on nonclinical data in syngeneic mouse tumour models (see WO 2018/185135).

Other PD-1 axis inhibitors known in the art are pembrolizumab (anti-PD-1), nivolumab (anti-PD-1), pidilizumab (anti-PD-1), tislelizumab (anti-PD-1), spartalizumab (anti-PD-1), durvalumab (anti-PD-L1/L2), atezolizumab (anti-PD-L1/L2), avelumab (anti-PD-L1/L2), toripalimab (anti-PD-L1/L2), cemiplimab (anti-PD-L1/L2), camrelizumab (anti-PD-L1/L2), dostarlimab (anti-PD-L1/L2) and cetrelimab (anti-PD-L1/L2).

### 2. DETAILS OF THE INVENTION

### Figures

FIG. 1 is a swimmer plot for tumor response to treatment with the MDM2-antagonist of formula I depending on the patient's MDM2-amplification status for liposarcoma patients only in trial 1403-0001 (WDLPS=well differentiated liposarcoma, DDLPS=dedifferentiated liposarcoma). Each column represents the treatment of one individual patient.

FIG. 2 shows the BLRM sensitivity analysis (based on the adverse events throughout all treatment cycles) in all patients treated in arm A of study 1403-0001: the 45 mg dose is the largest dose fulfilling the EWOC criteria.

FIG. 3 is a waterfall plot for tumor shrinkage in response to treatment with the MDM2-antagonist of formula I alone or in combination with ezabenlimab and anti-LAG-3 for all patients in studies 1403-0001 and 1403-0002 depending on the patient's MDM2 amplification status. Each column represents the treatment of one individual patient.

FIG. 4 is a swimmer plot for tumor response to treatment with the MDM2-antagonist of formula I depending on the patient's MDM2-amplification status for all patients in trial 1403-0001. Each column represents the treatment of one individual patient.

FIG. 5 is a plot showing tumor response to treatment with the MDM2-antagonist of formula I in combination with the PD-1 antibody ezabenlimab (marked by “D” for double combination) and in combination with the PD-1 antibody ezabenlimab and the anti-LAG 3 antibody BI 754111 (marked by “T” for triple combination) for study 1403-0002. For the double combination the MDM2-inhibitor of formula I has been dosed orally in the 30 mg/45 mg dose D1/q3w (10 patients/5 patients) and ezabenlimab has been dosed intravenously 240 mg D1/q3w.

The lower panel is a waterfall plot for tumor shrinkage in response to treatment and the upper panel is swimmer plot for duration of tumor response to treatment.

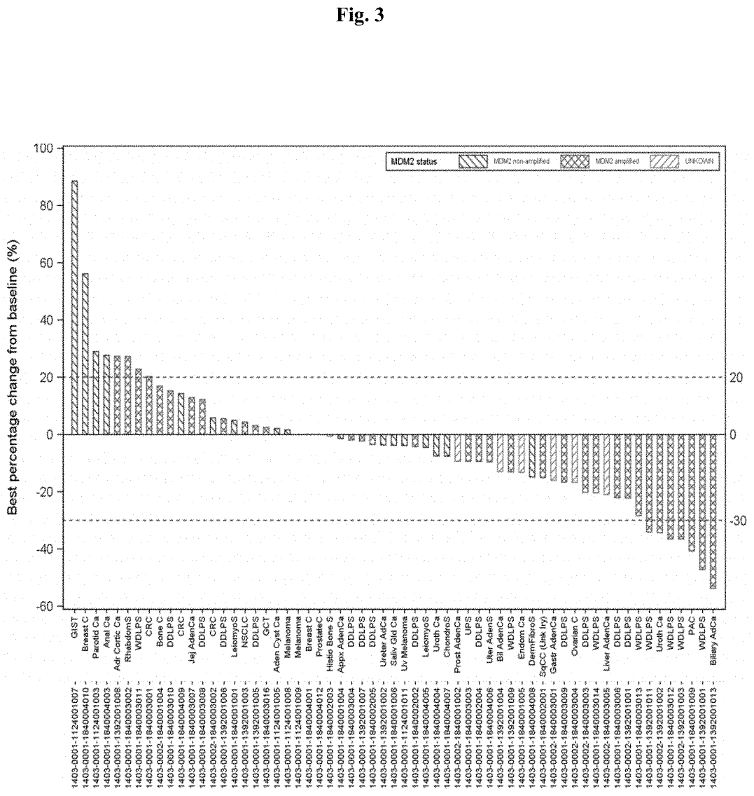

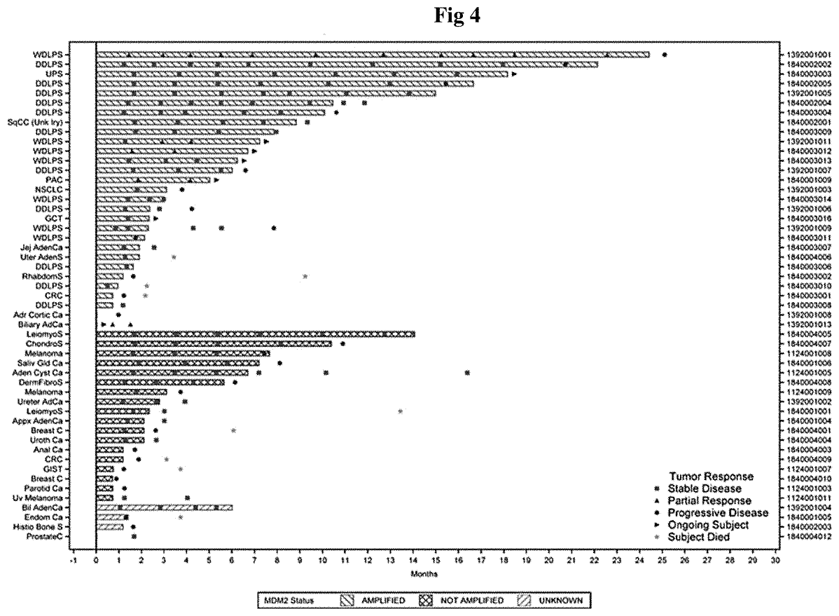

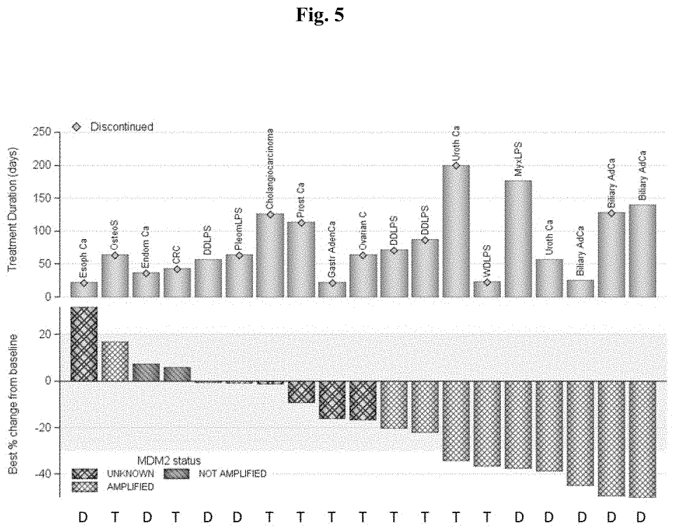

The abbreviations in FIG. 3, in FIG. 4 and in FIG. 5 mean the following:

| Abbreviation | Cancer form as meant by the abbreviation |
|---|---|
| DDLPS | Dedifferentiated liposarcoma |
| WDLPS | Well differentiated liposarcoma |
| GIST | Gastrointestinal Stromal tumor |
| Breast C | Breast cancer |
| Parotid Ca | Acinic cell carcinoma (Parotid carcinoma) |
| Anal Ca | Anal carcinoma (Sqamous cell carcinoma) |
| Adr Cortic Ca | Adrenal Cortic Carcinoma |
| Rhab odomS | Rhabdomyosarcoma |
| CRC | Colorectal colon carcinoma (adenocarcinoma) |
| Bone C | Bone Cancer |
| Jej AdenCA | Jejunal Adenosarcoma |
| LeiomyoS | Leiomyosarcoma |
| NSCLC | Non-small cell lung cancer |
| GCT | Germ Cell Tumor |
| Aden Cyst Ca | Adenoid Cystic Carcinoma (head and neck) |
| Melanoma | Melanoma |
| Prostata C | Prostate Cancer (Adenosarcoma) |
| Histio Bone S | Histio Bone Sarcoma |
| Appx AdenCa | Appendix Adenocarcinoma |
| Ureter AdCa | Ureter Adenocarcinoma |
| Saliv Gid Ca | Salivary Gland Carcinoma |
| Uv Melanoma | Uveal melanoma (ocular) (metastatic malignant |
| | melanoma) |
| Uroth Ca | Urothelial Carcinoma |
| Chondro S | Chondrosarcoma |
| Prost AdenCa | Prostate Adenosarcoma |
| UPS | Undifferentiated pleomorphic sarcoma |
| Uter AdenS | Uterine Adenosarcoma |
| Bil AdenCa (or | Biliary tract Adenocarcinoma |
| Biliary AdenCa | |
| or Biliary AdCa) | |
| Biliary Ca | Biliary tract carcinoma |
| Endom Ca | Endometrial Carcinoma |
| DermFibroS | Dermatofibrosarcoma |
| SqCC (Unk Iry) | Squamous carcinoma of unknown primary |
| Gast AdenCa | Gastric Adenocarcinoma |
| Ovarian C | Ovarian Cancer |
| Liver AdenCa | Liver Adenocarcinoma |
| PAC | Pancreatic cancer |
| MyxLPS | Myxoid liposarcoma |
| PleomLPS | Pleomorphic liposarcoma |
| OsteoS | Osteosarcoma |
| Esoph Ca | Esophageal carcinoma |

The MDM2-antagonist of formula I was—between many other structurally similar MDM2 antagonists—first described in WO 2017/060431 as a new treatment option in a large variety of different cancers. However, WO 2017/060431 does not show any clinical data, in particular no safety data, no efficacy data or pharmacological data from clinical trials in humans. Furthermore, WO 2017/060431 is also silent about efficacious doses and dose regimens in order to treat specific human cancers.

Consequently it was the aim of the present invention to provide doses and dose regimens of the specific MDM2-antagonist of formula I which lead to a satisfying treatment efficacy in cancer patients, preferably in soft tissue sarcoma patients, in particular in liposarcoma patients, while showing acceptable patient safety.

The results of the human Phase Ia/Ib trials in Section 3 show that a dose range of 30 mg to 45 mg of the MDM2-antagonist of formula I

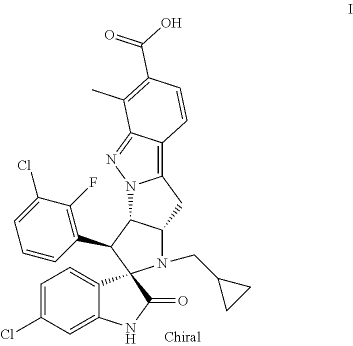 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

which is administered orally in a treatment cycle of once every three weeks (D1 q3w) and wherein this treatment cycle of once every three weeks (D1 q3w) may be repeated that many times as considered beneficial for the patient from a medical point of view, leads to an acceptable safety and to convincing results with respect to therapeutic efficacy in liposarcoma patients.

The acceptable safety of the above-mentioned doses and dose regimen can be concluded from the BLRM sensitivity analysis, which showed that both, the 30 mg dose and the 45 mg dose, fulfilled the EWOC criterion for Arm A. The 45 mg dose was the highest dose fulfilling the EWOC criterion for Arm A.

Additionally, the 45 mg dose of the MDM2-antagonist of formula I showed in the clinical trial 1403-0001 also clear signs of activity in two dedifferentiated liposarcoma (DDLPS) patients with a disease stabilization:
a first DDLPS patient treated with the MDM2-antagonist of formula I as a third line therapy showed a tumor shrinkage of approx. 10% for 353 days (see Table 4) and
a second DDLPS patient treated with the MDM2-antagonist of formula I as a second line therapy showed a tumor shrinkage of approx. 5% for 709 days (see Table 4).

In contrast to that, first line therapy with doxorubicin has been reported to lead only to a median Progression-free Survival (mPFS) between 2 and 4 months (Italiano et al, Ann. Oncol. (2012), Vol. 23, No. 6, pp. 1601-1607, Savina et al, BMC Medicine (2017), Vol. 15, pp. 78, Langmans et al, Oncol. Res. Treat. (2019), Vol. 42, pp. 396-403).

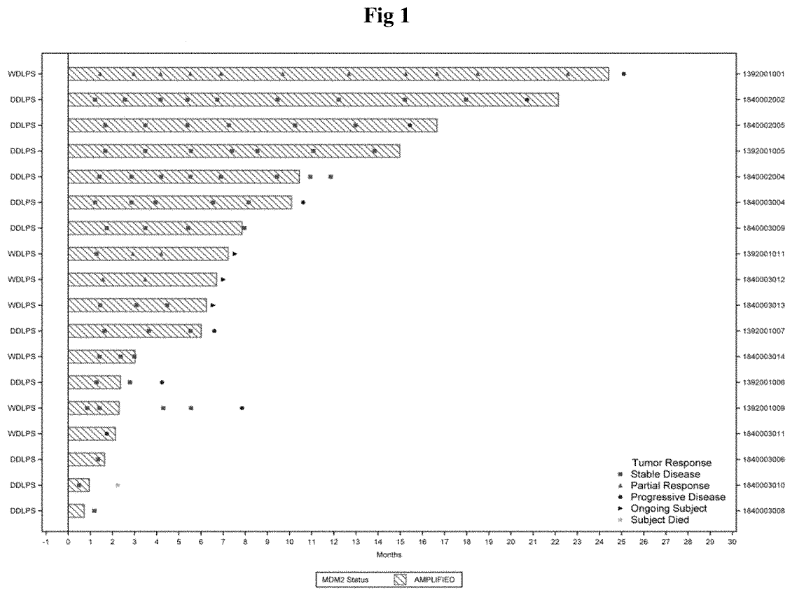

Furthermore, the results of the clinical trial 1403-0001 also show a convincing therapeutic efficacy of the MDM2-antagonist of formula I in well differentiated liposarcoma (WDLPS) patients. From seven well differentiated liposarcoma patients in trial 1403-0001 three experienced a partial response after treatment with the MDM2-antagonist of formula I (as shown in FIG. 1 and in FIG. 4).

Additionally, the results of the clinical trial 1403-0001 also show a convincing therapeutic efficacy of the MDM2-antagonist of formula I in other p53 wildtype and MDM2-amplified non-sarcoma cancer forms: for example one patient with a P53-wildtype, MDM2-amplified adenocarcinoma showed a partial response with 54% tumor shrinkage (see last patient from FIG. 3 “Biliary AdCA” from study 1403-0001) and a further patient with a p53 wildtype, MDM2-amplified pancreatic adenocarcinoma (PAC) showed a partial response of 41% tumor shrinkage (see third last patient from FIG. 3 “PAC” from study 1403-0001).

Preliminary signs of therapeutic efficacy for the combination trial 1403-0002 (combination of the MDM2-inhibitor of formula I with ezabenlimab and anti-LAG-3) could also be observed for one patient with MDM2-amplified well differentiated liposarcoma (WDLPS) with a partial response (see FIG. 3, fourth-last patient from the 1403-0002 study) and for one patient with an MDM2-amplified urothelial carcinoma with a partial response (see sixth-last patient from FIG. 3 “Uroth Ca” for the 1403-0002 study).

Furthermore, two dedifferentiated liposarcoma (DDLPS) patients (one DDLPS patient on arm A with the dosing regimen D1 q3w of the trial 1403-0001 (see Table 4) and another DDLPS patient on arm B with the dosing regimen D1, D8 28 days of the trial 1403-0001 (see Table 5)), which both had no prior history of systemic therapy, experienced long disease stabilizations and stayed on treatment for 342 and 542 days, respectively. This shows that treatment with the MDM2 antagonist of formula I is also suitable for “first line treatment” or “primary treatment” of dedifferentiated liposarcoma patients.

Furthermore, three patients of the trial 1403-0001 with cancers that were determined to be P53-wildtype and “non-MDM2-amplified” (see FIG. 4) showed surprisingly a disease stabilization (SD) for more than 7 months: so for instance in
one patient with “non-MDM2 amplified leiomyosarcoma” showed a disease stabilization for more than 14 months by treatment with the MDM2-antagonist of formula I (FIG. 4)
one patient with “non-MDM2-amplified chondrosarcoma” showed a disease stabilization for more than 10 months by treatment with the MDM2-antagonist of formula I (FIG. 4) and
one patient with “non-MDM2-amplified melanoma” showed a disease stabilization for more than 7 months by treatment with the MDM2-antagonist of formula I (FIG. 4).

This shows that the MDM2-antagonist of formula I is also suitable to treat P53-wildtype and “non-MDM2-amplified cancer types”. This finding is rather surprising, since MDM2 amplification is in the scientific community broadly accepted as the dominant mechanism through which human tumors raise MDM2 levels to abrogate p53 wildtype function (see Oliner et al, Cold Spring Harb Perspect Med, 2015M 6:a026336).

Consequently the invention refers to an oral pharmaceutical composition comprising the MDM2-antagonist of formula I

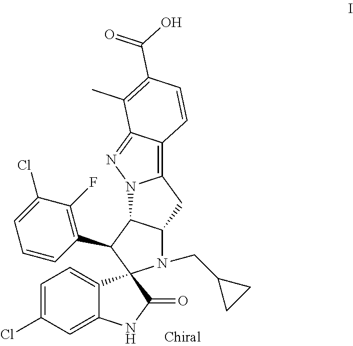 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

in a dose range of 30 mg to 45 mg for use in the treatment of cancer, wherein this oral pharmaceutical composition is administered in a treatment cycle of once every three weeks (D1 q3w), wherein this treatment cycle of once every three weeks (D1 q3w) may be repeated that many times as considered beneficial for the patient from a medical point of view.

For example this treatment cycle of once every three weeks (D1 q3w) may be repeated 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 times or even more often. The number of repetitions of the once every three weeks (D1 q3w) treatment cycle is generally considered beneficial for the patient from a medical point of view as long as the treatment stays tolerable with respect to the side effects and as long as the treatment stays efficacious that means it leads at least to a disease stabilization (and no progression of the disease).

In a further preferred embodiment the invention relates to the above-mentioned oral pharmaceutical composition, wherein the cancer that is treated is a p53 wildtype form of cancer.

In another preferred embodiment the invention refers to the above-mentioned oral pharmaceutical composition, wherein the cancer that is treated is a soft tissue sarcoma.

In a further preferred embodiment the invention relates to the above-mentioned oral pharmaceutical composition, wherein the cancer that is treated is liposarcoma.

In another preferred embodiment the invention refers to the above-mentioned oral pharmaceutical composition, wherein the cancer that is treated is well differentiated liposarcoma (WDLPS) or dedifferentiated liposarcoma (DDLPS).

In a further preferred embodiment the invention relates to the above-mentioned oral pharmaceutical composition, wherein the cancer that is treated is dedifferentiated liposarcoma (DDLPS).

In a further preferred embodiment the invention relates to the above-mentioned oral pharmaceutical composition, wherein the cancer that is treated is well differentiated liposarcoma (WDLPS).

In a further preferred embodiment the invention relates to the above-mentioned oral pharmaceutical composition, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned oral pharmaceutical composition, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned oral pharmaceutical composition, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned oral pharmaceutical composition, wherein the cancer that is treated is selected from the group consisting of biliary tract cancer and biliary adenocarcinoma.

In another preferred embodiment the invention refers to the above-mentioned oral pharmaceutical composition, wherein the MDM2-antagonist of formula I is present in a 45 mg dose.

In a further preferred embodiment the invention relates to the above-mentioned oral pharmaceutical composition, wherein the MDM2-antagonist of formula I is present in a 30 mg dose.

The invention relates in a further embodiment to a method for the treatment of cancer comprising administering a therapeutically effective amount of the MDM2-antagonist of formula I

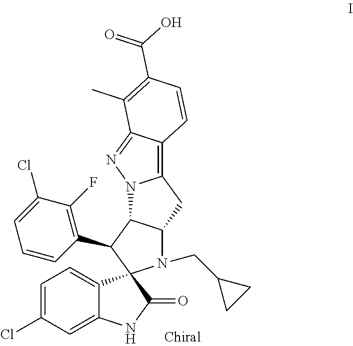 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

to a patient in need of such treatment, wherein the MDM2-antagonist is administered in a dose range of 30 mg to 45 mg in a treatment cycle of once every three weeks (D1 q3w) and wherein this treatment cycle of once every three weeks (D1 q3w) may be repeated that many times as considered beneficial for the patient from a medical point of view.

In another preferred embodiment the invention refers to the above-mentioned method, wherein the cancer that is treated is a p53 wildtype form of cancer.

In a further preferred embodiment the invention relates to the above-mentioned method, wherein the cancer that is treated is a soft tissue sarcoma.

In another preferred embodiment the invention refers to the above-mentioned method, wherein the cancer that is treated is liposarcoma.

In a further preferred embodiment the invention relates to the above-mentioned method, wherein the cancer that is treated is well differentiated liposarcoma (WDLPS) or dedifferentiated liposarcoma (DDLPS).

In another preferred embodiment the invention refers to the above-mentioned method, wherein the cancer that is treated is dedifferentiated liposarcoma (DDLPS).

In another preferred embodiment the invention refers to the above-mentioned method, wherein the cancer that is treated is well differentiated liposarcoma (WDLPS).

In a further preferred embodiment the invention relates to the above-mentioned method, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned method, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned method, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned method, wherein the cancer that is treated is selected from the group consisting of biliary tract cancer and biliary adenocarcinoma.

In a further preferred embodiment the invention relates to the above-mentioned method, wherein the MDM2-antagonist of formula I is present in a 45 mg dose.

In another preferred embodiment the invention refers to the above-mentioned method, wherein the MDM2-antagonist of formula I is present in a 30 mg dose.

The invention relates in a further embodiment to the use of a dose range of 30 mg to 45 mg of the compound of formula I

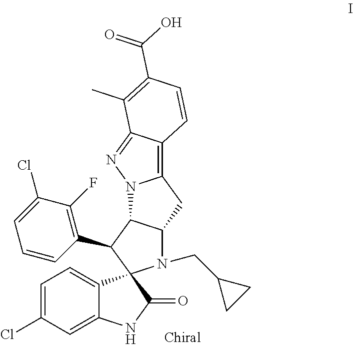 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for the manufacture of an oral pharmaceutical composition for the treatment of cancer that is administered in a treatment cycle of once every three weeks (D1 q3w), wherein this treatment cycle of once every three weeks (D1 q3w) may be repeated that many times as considered beneficial for the patient from a medical point of view.

In another preferred embodiment the invention refers to the above-mentioned use, wherein the cancer that is treated is a p53 wildtype form of cancer.

In a further preferred embodiment the invention relates to the above-mentioned use, wherein the cancer that is treated is a soft tissue sarcoma.

In another preferred embodiment the invention refers to the above-mentioned use, wherein the cancer that is treated is liposarcoma.

In a further preferred embodiment the invention relates to the above-mentioned use, wherein the cancer that is treated is well differentiated liposarcoma (WDLPS) or dedifferentiated liposarcoma (DDLPS).

In another preferred embodiment the invention refers to the above-mentioned use, wherein the cancer that is treated is dedifferentiated liposarcoma (DDLPS).

In another preferred embodiment the invention refers to the above-mentioned use, wherein the cancer that is treated is well differentiated liposarcoma (WDLPS).

In a further preferred embodiment the invention relates to the above-mentioned use, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned use, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned use, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned use, wherein the cancer that is treated is selected from the group consisting of biliary tract cancer and biliary adenocarcinoma.

In a further preferred embodiment the invention relates to the above-mentioned use, wherein the MDM2-antagonist of formula I is present in a 45 mg dose.

In another preferred embodiment the invention refers to the above-mentioned use, wherein the MDM2-antagonist of formula I is present in a 30 mg dose.

The invention refers in another embodiment to an MDM2-antagonist of formula I

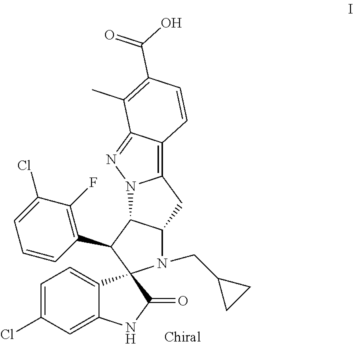 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for its use in first line systemic treatment (primary treatment) of dedifferentiated liposarcoma (DDLPS).

In a further preferred embodiment the invention relates to the above-mentioned MDM2-antagonist of formula I for its use in first line systemic treatment of dedifferentiated liposarcoma (DDLPS), wherein the MDM2-antagonist of formula I is administered to the patient in need thereof in a treatment cycle of once every three weeks (D1 q3w), wherein this treatment cycle may be repeated that many times as considered beneficial for the patient from a medical point of view.

In another preferred embodiment the invention refers to the above-mentioned MDM2-antagonist of formula I for its use in first line systemic treatment of dedifferentiated liposarcoma (DDLPS), wherein the MDM2-antagonist is administered to the patient in each treatment cycle in a dose range of 30 mg to 45 mg.

In a further preferred embodiment the invention relates to the above-mentioned MDM2-antagonist of formula I for its use in first line systemic treatment of dedifferentiated liposarcoma (DDLPS), wherein the MDM2-antagonist is administered to the patient in each treatment cycle in a dose of 45 mg.

In another preferred embodiment the invention refers to the above-mentioned MDM2-antagonist of formula I for its use in first line systemic treatment of dedifferentiated liposarcoma (DDLPS), wherein the MDM2-antagonist is administered to the patient in each treatment cycle in a dose of 30 mg.

The invention relates in a further embodiment to the use of the MDM2-antagonist of formula I

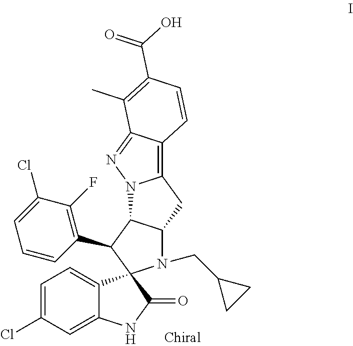 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for the manufacture of a medicament for the first line systemic chemotherapeutic treatment (primary treatment) of dedifferentiated liposarcoma (DDLPS).

In another preferred embodiment the invention refers to the above-mentioned use of the MDM2-antagonist of formula I for the manufacture of a medicament for the first line systemic chemotherapeutic treatment (primary treatment) of dedifferentiated liposarcoma (DDLPS), wherein the MDM2-antagonist of formula I is administered to the patient in need thereof in a treatment cycle of once every three weeks (D1 q3w), wherein this treatment cycle may be repeated that many times as considered beneficial for the patient from a medical point of view.

In a further preferred embodiment the invention relates to the above-mentioned use of the MDM2-antagonist of formula I for the manufacture of a medicament for the first line systemic treatment (primary treatment) of dedifferentiated liposarcoma (DDLPS), wherein the MDM2-antagonist is administered to the patient in each treatment cycle in a dose range of 30 mg to 45 mg.

In another preferred embodiment the invention refers to the above-mentioned use of the MDM2-antagonist of formula I for the manufacture of a medicament for the first line systemic treatment (primary treatment) of dedifferentiated liposarcoma (DDLPS), wherein the MDM2-antagonist is administered to the patient in each treatment cycle in a dose of 45 mg.

In a further preferred embodiment the invention relates to the above-mentioned use of the MDM2-antagonist of formula I for the manufacture of a medicament for the first line systemic treatment (primary treatment) of dedifferentiated liposarcoma (DDLPS), wherein the MDM2-antagonist is administered to the patient in each treatment cycle in a dose of 30 mg.

The invention relates in a further embodiment to the use of a combination of 45 mg of the MDM2-antagonist of formula I

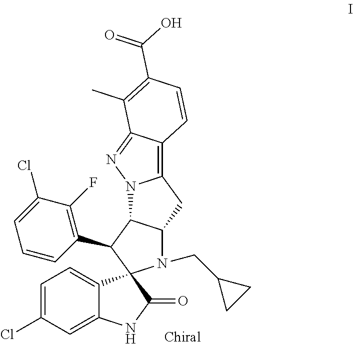 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

and of 240 mg ezabenlimab for the treatment of cancer, wherein both, the MDM2-antagonist of formula I and ezabenlimab are administered to the patient in need thereof in a treatment cycle of once every three weeks (D1 q3w), wherein this treatment cycle may be repeated that many times as considered beneficial for the patient from a medical point of view.

In a further preferred embodiment the invention relates to the above-mentioned combined use of 45 mg of the MDM2-antagonist of formula I and of 240 mg ezabenlimab for the treatment of cancer, wherein the cancer is a p53 wildtype form of cancer.

In another preferred embodiment the invention refers to the above-mentioned combined use of 45 mg of the MDM2-antagonist of formula I and of 240 mg ezabenlimab for the treatment of cancer, wherein the cancer is a soft tissue sarcoma.

In a further preferred embodiment the invention relates to the above-mentioned combined use of 45 mg of the MDM2-antagonist of formula I and of 240 mg ezabenlimab for the treatment of cancer, wherein the cancer is liposarcoma.

In another preferred embodiment the invention refers to the above-mentioned combined use of 45 mg of the MDM2-antagonist of formula I and of 240 mg ezabenlimab for the treatment of cancer, wherein the cancer is a liposarcoma selected from the group consisting of a dedifferentiated liposarcoma (DDLPS) and a well differentiated liposarcoma (WDLPS).

In a further preferred embodiment the invention relates to the above-mentioned combined use of 45 mg of the MDM2-antagonist of formula I and of 240 mg ezabenlimab for the treatment of cancer, wherein the cancer is dedifferentiated liposarcoma (DDLPS).

In another preferred embodiment the invention refers to the above-mentioned combined use of 45 mg of the MDM2-antagonist of formula I and of 240 mg ezabenlimab for the first line treatment of cancer.

In a further preferred embodiment the invention relates to the above-mentioned combined use of 45 mg of the MDM2-antagonist of formula I and of 240 mg ezabenlimab for the treatment of cancer, preferably liposarcoma, more preferably dedifferentiated liposarcoma (DDLPS), wherein the 45 mg of the MDM2-antagonist of formula I is administered orally and wherein the 240 mg ezabenlimab is administered intravenously.

The invention relates in a further embodiment to an MDM2-antagonist of formula I

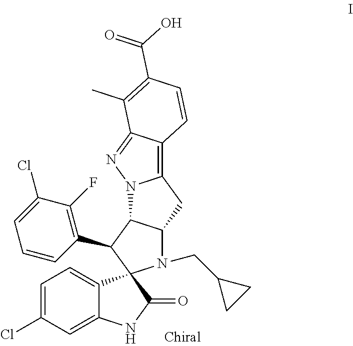 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for use in the treatment of cancer, wherein the MDM2-antagonist of formula I is administered in combination with ezabenlimab and wherein
both the MDM2-antagonist and ezabenlimab are administered to the patient in need thereof in a treatment cycle of once every three weeks (D1 q3w);
the MDM2-antagonist is administered in a dose of 45 mg;
ezabenlimab is administered in a dose of 240 mg; and
this treatment cycle may be repeated that many times as considered beneficial for the patient from a medical point of view.

In a further preferred embodiment the invention relates to the above-mentioned MDM2-antagonist of formula I for use in the treatment of cancer, wherein the cancer is a p53 wildtype form of cancer.

In another preferred embodiment the invention refers to the above-mentioned MDM2-antagonist of formula I for use in the treatment of cancer, wherein the cancer is a soft tissue sarcoma.

In a further preferred embodiment the invention relates to the above-mentioned MDM2-antagonist of formula I for use in the treatment of cancer, wherein the cancer is liposarcoma.

In another preferred embodiment the invention refers to the above-mentioned MDM2-antagonist of formula I for use in the treatment of cancer, wherein the cancer is a liposarcoma selected from the group consisting of a dedifferentiated liposarcoma (DDLPS) and a well differentiated liposarcoma (WDLPS).

In a further preferred embodiment the invention relates to the above-mentioned MDM2-antagonist of formula I for use in the treatment of cancer, wherein the cancer is dedifferentiated liposarcoma (DDLPS).

In a further preferred embodiment the invention relates to the above-mentioned MDM2-antagonist of formula I for use in the treatment of cancer, wherein the cancer is well differentiated liposarcoma (WDLPS).

In a further preferred embodiment the invention relates to the above-mentioned MDM2-antagonist of formula I for use in the treatment of cancer, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned MDM2-antagonist of formula I for use in the treatment of cancer, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned MDM2-antagonist of formula I for use in the treatment of cancer, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned MDM2-antagonist of formula I for use in the treatment of cancer, wherein the cancer that is treated is selected from the group consisting of biliary tract cancer and biliary adenocarcinoma.

In another preferred embodiment the invention refers to the above-mentioned MDM2-antagonist of formula I for use in the treatment of cancer, wherein the treatment of cancer is a first line treatment.

In a further preferred embodiment the invention relates to the above-mentioned MDM2-antagonist of formula I for use in the treatment of cancer, wherein the 45 mg of the MDM2-antagonist of formula I is administered orally and wherein the 240 mg ezabenlimab is administered intravenously.

The invention relates in a further embodiment to a method for the treatment of cancer comprising administering a therapeutically effective amount of the MDM2-antagonist of formula I

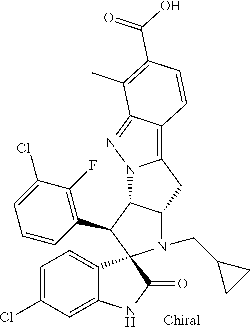 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

to a patient in need of such treatment, wherein the MDM2-antagonist of formula I is administered in combination with ezabenlimab and wherein
both the MDM2-antagonist and ezabenlimab are administered to the patient in a treatment cycle of once every three weeks (D1 q3w);
the MDM2-antagonist is administered in a dose of 45 mg;
ezabenlimab is administered in a dose of 240 mg; and
this treatment cycle may be repeated that many times as considered beneficial for the patient from a medical point of view.

In another preferred embodiment the invention refers to the above-mentioned method, wherein the cancer that is treated is a p53 wildtype form of cancer.

In a further preferred embodiment the invention relates to the above-mentioned method, wherein the cancer that is treated is a soft tissue sarcoma.

In another preferred embodiment the invention refers to the above-mentioned method, wherein the cancer that is treated is liposarcoma.

In a further preferred embodiment the invention relates to the above-mentioned method, wherein the cancer that is treated is well differentiated liposarcoma (WDLPS) or dedifferentiated liposarcoma (DDLPS).

In another preferred embodiment the invention refers to the above-mentioned method, wherein the cancer that is treated is dedifferentiated liposarcoma (DDLPS).

In another preferred embodiment the invention refers to the above-mentioned method, wherein the cancer that is treated is well differentiated liposarcoma (WDLPS).

In a further preferred embodiment the invention relates to the above-mentioned method, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned method, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned method, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned method, wherein the cancer that is treated is selected from the group consisting of biliary tract cancer and biliary adenocarcinoma.

In another preferred embodiment the invention refers to the above-mentioned method, wherein the treatment of cancer is a first line treatment.

In a further preferred embodiment the invention relates to the above-mentioned method, wherein the 45 mg of the MDM2-antagonist of formula I is administered orally and wherein the 240 mg ezabenlimab is administered intravenously.

In further preferred embodiments of the invention ezabenlimab is replaced in the embodiments disclosed hereinbefore by an alternative PD-1 axis inhibitor selected from the group consisting of pembrolizumab, nivolumab, pidilizumab, tislelizumab, spartalizumab, durvalumab, atezolizumab, avelumab, toripalimab, cemiplimab, camrelizumab, dostarlimab and cetrelimab. Most preferred ezabenlimab is replaced by pembrolizumab or nivolumab in these embodiments.

It should be understood that the MDM2-antagonist of formula I and ezabenlimab or another PD-1 axis inhibitor as disclosed herein are usually administered at the same day 1 of a treatment cycle, i.e. are administered concomitantly or concurrently the same day. A separate or consecutive or staggered administration of both drugs on different days 1 however may also be possible. It should also be understood that if ezabenlimab is replaced in the embodiments by an alternative PD-1 axis inhibitor, both as disclosed hereinbefore, then the alternative PD-1 axis inhibitor is usually dosed in the same amount that it is dosed and approved for in monotherapy or in other combination treatments.

The invention relates in a further embodiment to the use of the MDM2-antagonist of formula I

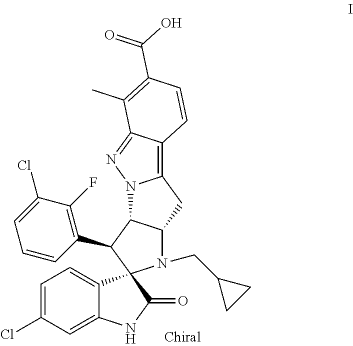 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for the manufacture of a medicament for the treatment of a p53-wildtype and non-MDM2-amplified form of cancer.

In another preferred embodiment the invention refers to the above-mentioned use of the MDM2-antagonist of formula I for the manufacture of a medicament for the treatment of a p53-wildtype and non-MDM2-amplified form of cancer, wherein this p53 wildtype and non-MDM2-amplified form of cancer is selected from the group consisting of leiomyosarcoma, chondrosarcoma and melanoma.

In a further preferred embodiment the invention relates to the above-mentioned use of the MDM2-antagonist of formula I for the manufacture of a medicament for the treatment of a p53-wildtype and non-MDM2-amplified form of cancer, wherein 45 mg of the MDM2-antagonist of formula I is administered to the patient in need thereof in a treatment cycle of once every three weeks (D1q3w), wherein this treatment cycle may be repeated that many times as considered beneficial for the patient from a medical point of view.

In a further preferred embodiment the invention relates to the above-mentioned use of the MDM2-antagonist of formula I for the manufacture of a medicament for the treatment of a p53-wildtype and non-MDM2-amplified form of cancer, wherein
the MDM2-antagonist is administered to the patient in need thereof in a treatment cycle of once every three weeks (D1 q3w);
the MDM2-antagonist is administered in a dose of 45 mg;
this treatment cycle may be repeated that many times as considered beneficial for the patient from a medical point of view.

The invention relates in a further embodiment to the MDM2-antagonist of formula I

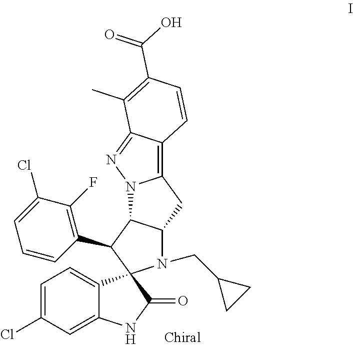 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

for use in the treatment of a p53-wildtype and non-MDM2-amplified form of cancer.

In another preferred embodiment the invention refers to the above-mentioned MDM2-antagonist of formula I for use in the treatment of a p53-wildtype and non-MDM2-amplified form of cancer, wherein this p53 wildtype and non-MDM2-amplified form of cancer is selected from the group consisting of leiomyosarcoma, chondrosarcoma and melanoma.

In a further preferred embodiment the invention relates to the above-mentioned MDM2-antagonist of formula I for use in the treatment of a p53-wildtype and non-MDM2-amplified form of cancer, wherein the cancer that is treated is selected from the group consisting of high-grade leiomyosarcoma, leiomyosarcoma, gastrointestinal stromal tumour (GIST), adenosarcoma, dermatofibrosarcoma, osteosarcoma, rhabdomyosarcoma, undifferentiated pleomorphic sarcoma (UPS), chondrosarcoma, myxoid chondrosarcoma, myxoid liposarcoma, myxofibrosarcoma, synovial sarcoma and uterine adenosarcoma.

In a further preferred embodiment the invention relates to the above-mentioned MDM2-antagonist of formula I for use in the treatment of a p53-wildtype and non-MDM2-amplified form of cancer, wherein the cancer that is treated is selected from the group consisting of biliary tract cancer, biliary adenocarcinoma, pancreatic adenocarcinoma, urothelial carcinoma, intrahepatic cholangiocarcinoma, gastric adenocarcinoma, non-small cell lung cancer (NSCLC), endometrial cancer, colorectal cancer, glioblastoma and melanoma.

In a further preferred embodiment the invention relates to the above-mentioned MDM2-antagonist of formula I for use in the treatment of a p53-wildtype and non-MDM2-amplified form of cancer, wherein the cancer that is treated is selected from the group consisting of biliary tract cancer, intrahepatic cholangiocarcinoma, extrahepatic cholangiocarcinoma, gall bladder carcinoma and ampullary carcinoma.

In a further preferred embodiment the invention relates to the above-mentioned MDM2-antagonist of formula I for use in the treatment of a p53-wildtype and non-MDM2-amplified form of cancer, wherein
the MDM2-antagonist is administered to the patient in need thereof in a treatment cycle of once every three weeks (D1 q3w);
the MDM2-antagonist is administered in a dose of 45 mg;
this treatment cycle may be repeated that many times as considered beneficial for the patient from a medical point of view.

### 3. RESULTS

### 3.1 Trials

### 3.1.1 Trial 1403-0001

Trial 1403-0001 was a first in human Phase Ia/Ib, open label, multicenter, dose-escalation study of the MDM2-antagonist of formula I in patients with advanced or metastatic solid tumors. The Phase Ia dose escalation part aimed to define the Maximum Tolerated Dose (MTD) and the Recommended Dose for Expansion (RDE) for the MDM2-antagonist of formula I based on safety and tolerability, pharmacokinetics, pharmacodynamics, and preliminary efficacy. The RDE will be further explored in the Phase Ib expansion part of the trial.

Fifty-four patients had been treated with the MDM2-antagonist of formula I in the Phase Ia dose escalation part of clinical trial 1403-0001. Twenty-nine patients were treated in Arm A (dosing regimen: dosing on Day 1 of a 21 days-cycle, D1 q3w) and twenty-five patients were treated in Arm B (dosing regimen: dosing on Day 1 and on Day 8 of a 28 days-cycle, D1 D8 q4w). Dose levels evaluated in Arm A were 10, 20, 30, 45, 50, 60, and 80 mg. Dose levels evaluated in Arm B (referring to the dose administered on Day 1 and Day 8) were 5, 10, 15, 20, 30, 45, and 60 mg.

Arm B (referring to the dose administered on Day 1 and Day 8) were 5, 10, 15, 20, 30, 45, and 60 mg.

Based on exploratory safety and efficacy analyses, for Arm A, 60 mg q3w was selected as the MTD and 45 mg D1 q3w was selected as the RDE. For Arm B, 45 mg D1 D8 q4w was selected as the MTD. Preliminary pharmacokinetic (PK) data were also used as supportive information for the dose decision.

It was decided to continue the expansion phase (Phlb) with the Arm A schedule at 45 mg as this dose was the highest one fulfilling the Escalation with Overdose Control criterion (EWOC criterion) from a Bayesian Logistic Regression Model (BLRM) analysis, taking into account the adverse events of interest during the entire treatment period (and not only during the MTD evaluation period) as outlined in the corresponding section thereafter. In addition, at this 45 mg dose considered safe in the long term, the MDM2-antagonist of formula I showed, among others, clear signs of activity in two dedifferentiated liposarcoma patients with a disease stabilization for 353 and 709 days, respectively.

### 3.1.2 Trial 1403-0002

A second Phase Ia/Ib trial (1403-0002) was initiated to assess safety and tolerability of escalating doses of the MDM 2-antagonist of formula I (starting at 10 mg dose level) in combination with fixed flat doses of the anti-PD-1 monoclonal antibody ezabenlimab (in a fixed 240 mg dose) and anti-LAG-3 monoclonal antibody LAG3-1, as described in WO2018/185135 (in a fixed 600 mg dose) given on Day 1 every 21 days (D1q3w). This dose escalation with the above-mentioned triple combination was prematurely terminated and replaced with the double combination (the MDM2-antagonist of formula I+anti-PD-1 mAb ezabenlimab).

### 3.1.3 Dose Finding for the MDM2 Antagonist of Formula I in Monotherapy as Supported by the Results of Trial 1403-0001

### 3.1.3.1 Safety Summary for 1403-0001—Arm A

During the on-treatment period, all 29 patients were reported with adverse events (AEs). The most frequent AEs by preferred term (reported for >20% of patients) were nausea (82.8%), fatigue (51.7%), vomiting (48.3%), anemia and decreased appetite (41.4% each), platelet count decreased (37.9%), diarrhea (34.5%) and back pain (20.7%). A total of 27 patients (93.1%) were reported with at least 1 AE judged to be related to the MDM2-antagonist of formula I by investigators. The most common drug-related AEs (reported for >20% of patients) were nausea (72.4%), fatigue and vomiting (41.1% each), and platelet count decreased (37.9%), decreased appetite (34.5%), diarrhea and white blood cell count decreased (27.6% each), and anemia (24.1%).

There were 15 patients (51.7%) with an AE Grade 3. The most frequently reported Grade 3 AE was platelet count decreased (13.8%), and 5 patients (17.2%) had an AE of Grade 4. The most frequently reported Grade 4 AE was neutrophil count decreased (10.3%). No fatal AEs were reported.

There were 9 patients (31.0%) with on-treatment serious AEs (SAEs). SAEs reported for more than 1 patients were platelet count decreased (6.9%).

There were 9 patients (31.0%) with AEs leading to dose reduction. For 6 patients (20.7%) a dose reduction was required due to neutropenia and/or thrombocytopenia, for 2 patients (6.9%) due to nausea, and for 1 patient (3.4%) due to enterocolitis.

There were 2 patients (6.9%) with AEs leading to permanent treatment discontinuation, of whom 1 patient had AE of nausea Grade 3 judged to be drug-related by investigator, and 1 patient had AE of embolism arterial Grade 3 judged to be drug-related by investigator. Dose reductions were not conducted for either patient.

Dose limiting toxicities (DLTs) were reported across all dose levels for all cycles for 9 patients overall (31.0%). Thereof, 5 patients (17.2%) had DLTs during the MTD evaluation period (cycle 1). At dose level 45 mg, 1 patient had a DLT of nausea Grade 3 (day 8) requiring a dose reduction and therapy (outcome recovered), and 1 patient had a DLT of platelet count decreased Grade 3 (day 30) requiring a dose delay starting cycle 2 (outcome recovered). At dose level 60 mg, 1 patient had a DLT of enterocolitis Grade 3 (day 3) requiring a dose reduction and therapy (outcome recovered); and at dose level 80 mg, 1 patient had 2 DLTs of neutrophil count decreased Grade 4 and thrombocytopenia Grade 4 (both day 12) requiring a dose delay starting cycle 2, dose reduction and therapy (outcome both DLTs not recovered), and 1 patient had a DLT of thrombocytopenia Grade 4 (day 35) requiring a dose delay and therapy (outcome not recovered).

Table 1 shows a safety summary by dose for patients treated in the 1403-0001 trial—Arm A.

TABLE 1
Adverse event overall summary - Arm A - Treated Set

| | Comp I | | Comp. I | | Comp. I | | Comp. I | | Comp. I | | Comp. I | | Comp. I | | Comp. I | |
|---|---|---|---|---|---|---|---|---|---|---|---|---|---|---|---|---|
| | 10 mg | | 20 mg | | 30 mg | | 45 mg | | 50 mg | | 60 mg | | 80 mg | | Total | |
| | N | % | N | % | N | % | N | % | N | % | N | % | N | % | N | % |
| Number of subjects | 1 | 100 | 2 | 100 | 3 | 100 | 6 | 100 | 4 | 100 | 7 | 100 | 6 | 100 | 29 | 100 |
| Subjects with any AE | 1 | 100 | 2 | 100 | 3 | 100 | 6 | 100 | 4 | 100 | 7 | 100 | 6 | 100 | 29 | 100 |
| Patients with Dose | 1 | 100 | 1 | 50 | 0 | 0 | 2 | 33.3 | 0 | 0 | 1 | 14.3 | 4 | 66.7 | 9 | 31 |
| limiting toxicity | | | | | | | | | | | | | | | | |
| Subjects with | 1 | 100 | 2 | 100 | 3 | 100 | 5 | 83.3 | 3 | 75.0 | 7 | 100 | 6 | 100 | 27 | 93.1 |
| investigator defined | | | | | | | | | | | | | | | | |
| drug-related AEs | | | | | | | | | | | | | | | | |
| Subjects with AEs | 0 | 0 | 1 | 50 | 1 | 33.3 | 2 | 33.3 | 0 | 0 | 2 | 28.6 | 3 | 50 | 9 | 31 |
| leading to dose | | | | | | | | | | | | | | | | |
| reduction of trial drug | | | | | | | | | | | | | | | | |
| Subjects with AEs | 1 | 100 | 0 | 0 | 0 | 0 | 0 | 0 | 0 | 0 | 0 | 0 | 1 | 16.7 | 2 | 6.9 |
| leading to | | | | | | | | | | | | | | | | |
| Discontinuation of | | | | | | | | | | | | | | | | |
| trial drug | | | | | | | | | | | | | | | | |
| Dose limiting toxicity | 1 | 100 | 0 | 0 | 0 | 0 | 0 | 0 | 0 | 0 | 0 | 0 | 0 | 0 | 1 | 3.4 |
| Other adverse event | 0 | 0 | 0 | 0 | 0 | 0 | 0 | 0 | 0 | 0 | 0 | 0 | 1 | 16.7 | 1 | 3.4 |
| Subjects with serious | 1 | 100 | 1 | 50 | 0 | 0 | 0 | 0 | 2 | 50 | 3 | 42.9 | 2 | 33.3 | 9 | 31.0 |
| AEs | | | | | | | | | | | | | | | | |
| Results in Death | 0 | 0 | 0 | 0 | 0 | 0 | 0 | 0 | 0 | 0 | 0 | 0 | 0 | 0 | 0 | 0 |
| Is Life Threatening | 0 | 0 | 0 | 0 | 0 | 0 | 0 | 0 | 0 | 0 | 0 | 0 | 0 | 0 | 0 | 0 |
| Persists of Signif. | 0 | 0 | 0 | 0 | 0 | 0 | 0 | 0 | 0 | 0 | 0 | 0 | 0 | 0 | 0 | 0 |
| Disability/Incapacity | | | | | | | | | | | | | | | | |
| Requires or prolongs | 1 | 100 | 1 | 50 | 0 | 0 | 0 | 0 | 2 | 50 | 3 | 42.9 | 2 | 33.3 | 9 | 31.0 |
| Hospitalization | | | | | | | | | | | | | | | | |
| Cogenital Anomaly | 0 | 0 | 0 | 0 | 0 | 0 | 0 | 0 | 0 | 0 | 0 | 0 | 0 | 0 | 0 | 0 |
| or Birth Defect | | | | | | | | | | | | | | | | |
| Other Medically | 0 | 0 | 0 | 0 | 0 | 0 | 0 | 0 | 1 | 25.0 | 0 | 0 | 0 | 0 | 1 | 3.4 |
| Important Serious | | | | | | | | | | | | | | | | |
| Event | | | | | | | | | | | | | | | | |
| Subjects with | | | | | | | | | | | | | | | | |
| maximum CTC Grade | | | | | | | | | | | | | | | | |
| Grade 1 | 0 | 0 | 0 | 0 | 0 | 0 | 0 | 0 | 0 | 0 | 1 | 14.3 | 0 | 0 | 1 | 3.4 |
| Grade 2 | 0 | 0 | 1 | 50 | 1 | 33.3 | 3 | 50 | 0 | 0 | 1 | 14.3 | 2 | 33.3 | 8 | 27.6 |
| Grade 3 | 1 | 100 | 1 | 50 | 2 | 66.7 | 2 | 33.3 | 4 | 100 | 5 | 71.4 | 0 | 0 | 15 | 51.7 |
| Grade 4 | 0 | 0 | 0 | 0 | 0 | 0 | 1 | 16.7 | 0 | 0 | 0 | 0 | 4 | 66.7 | 5 | 17.2 |
| Grade 5 | 0 | 0 | 0 | 0 | 0 | 0 | 0 | 0 | 0 | 0 | 0 | 0 | 0 | 0 | 0 | 0 |

A subject may have serious AE(s) with multiple seriousness criteria.

Percentages are calculated using total number of subjects per treatment as the denominator.

MedDRA version used for reporting: 23.1

CTCAE v5.0 is used for reporting

### 3.1.3.2 Safety Summary for 1403-0001—Arm B

During the on-treatment period, all 25 patients were reported with adverse events (AEs). The most frequent AEs by preferred term (reported for >20% of patients) were nausea (92.0%), fatigue (52.0%), vomiting (64.0%), decreased appetite (40.0% each), platelet count decreased (28.0%), diarrhea (44.0%) and alopecia (24.0%). A total of 23 patients (92.0%) were reported with at least 1 AE judged to be related to the MDM2 antagonist of formula I by investigators. The most common drug-related AEs (reported for >20% of patients) were nausea (76.0%), vomiting (60.0%), fatigue (40.0%), diarrhea (32.0%), platelet count decreased and decreased appetite (28.0% each), and alopecia (24.0%).

There were 7 patients (28.0%) with an AE graded CTCAE Grade 3. The most frequently reported Grade 3 AE was anemia and platelet count decreased (8.0% each), and 4 patients (16.0%) had an AE of Grade 4. The most frequently reported Grade 4 AE was neutrophil count decreased platelet count decreased (8.0% each). No fatal AEs were reported.

There were 9 patients (36.0%) with on-treatment serious AEs (SAEs). SAEs reported for more than 1 patients were platelet count decreased (8.0%).

There were 6 patients (24%) with AEs leading to dose reduction. For 5 patients (20.0%) a dose reduction was required due to neutropenia and/or thrombocytopenia, and for 1 patient (4.0%) due to diarrhea.

There was 1 patient (4.0%) with 1 AE of vomiting Grade 1 (judged to be drug-related by investigator) leading to permanent treatment discontinuation. Dose reduction was not conducted for this patient, the dose was delayed due to this event in cycle 8.

Overall, for 3 patients (12.0%) DLTs were reported in the highest dosing regimens at 45 mg and 60 mg for all cycles. During the MTD evaluation period (cycle 1), 1 patient had a DLT of platelet count decreased Grade 4 (day 36) at dose level 45 mg requiring a dose delay starting cycle 2 (outcome recovered). At dose level 60 mg, 1 patient had 2 DLTs of neutrophil count decreased Grade 4 (day 31) and thrombocytopenia Grade 4 (day 29) requiring a dose delay starting cycle 2, dose reduction and therapy (outcome both DLTs not recovered), and 1 patient had 1 DLT of neutrophil count decreased Grade 3 (day 29) requiring dose delay, dose reduction and therapy (outcome not recovered).

Table 2 shows a safety summary by dose for patients treated in the 1403-0001 trial—Arm B.

TABLE 2
Adverse event overall summary - Arm B - Treated Set

| | Comp I | | Comp. I | | Comp. I | | Comp. I | | Comp. I | | Comp. I | | Comp. I | | Comp. I | |
|---|---|---|---|---|---|---|---|---|---|---|---|---|---|---|---|---|
| | 5 mg | | 10 mg | | 15 mg | | 20 mg | | 30 mg | | 45 mg | | 60 mg | | Total | |
| | N | % | N | % | N | % | N | % | N | % | N | % | N | % | N | % |
| Number of subjects | 3 | 100 | 3 | 100 | 3 | 100 | 3 | 100 | 4 | 100 | 6 | 100 | 3 | 100 | 25 | 100 |
| Subjects with any AE | 3 | 100 | 3 | 100 | 3 | 100 | 3 | 100 | 4 | 100 | 6 | 100 | 3 | 100 | 25 | 100 |
| Patients with Dose | 0 | 0 | 0 | 0 | 0 | 0 | 0 | 0 | 0 | 0 | 1 | 16.7 | 2 | 66.7 | 3 | 12.0 |
| limiting toxicity | | | | | | | | | | | | | | | | |
| Subjects with | 1 | 33.3 | 3 | 100 | 3 | 100 | 3 | 100 | 4 | 100 | 6 | 100 | 3 | 100 | 23 | 92.0 |
| investigator defined | | | | | | | | | | | | | | | | |
| drug-related AEs | | | | | | | | | | | | | | | | |
| Subjects with AEs | 0 | 0 | 1 | 33.3 | 0 | 0 | 0 | 0 | 2 | 50 | 1 | 16.7 | 2 | 66.7 | 6 | 24.0 |
| leading to dose | | | | | | | | | | | | | | | | |
| reduction of trial drug | | | | | | | | | | | | | | | | |
| Subjects with AEs | 0 | 0 | 0 | 0 | 0 | 0 | 0 | 0 | 0 | 0 | 1 | 16.7 | 0 | 0 | 1 | 4.0 |
| leading to | | | | | | | | | | | | | | | | |
| Discontinuation | | | | | | | | | | | | | | | | |
| of trial drug | | | | | | | | | | | | | | | | |
| Dose limiting toxicity | 0 | 0 | 0 | 0 | 0 | 0 | 0 | 0 | 0 | 0 | 0 | 0 | 0 | 0 | 0 | 0 |
| Other adverse event | 0 | 0 | 0 | 0 | 0 | 0 | 0 | 0 | 0 | 0 | 1 | 16.7 | 0 | 0 | 1 | 4.0 |
| Subjects with serious | 0 | 0 | 1 | 33.3 | 1 | 33.3 | 1 | 33.3 | 1 | 25.0 | 3 | 50.0 | 2 | 66.7 | 9 | 36.0 |
| AEs | | | | | | | | | | | | | | | | |
| Results in Death | 0 | 0 | 0 | 0 | 0 | 0 | 0 | 0 | 0 | 0 | 0 | 0 | 0 | 0 | 0 | 0 |
| Is Life Threatening | 0 | 0 | 0 | 0 | 0 | 0 | 0 | 0 | 0 | 0 | 0 | 0 | 0 | 0 | 0 | 0 |
| Persists of Signif. | 0 | 0 | 0 | 0 | 0 | 0 | 0 | 0 | 0 | 0 | 1 | 16.7 | 0 | 0 | 1 | 4.0 |
| Disability/Incapacity | | | | | | | | | | | | | | | | |
| Requires or Prolongs | 0 | 0 | 1 | 33.3 | 1 | 33.3 | 1 | 33.3 | 0 | 0 | 2 | 33.3 | 2 | 66.7 | 7 | 28.0 |
| Hospitalization | | | | | | | | | | | | | | | | |
| Cogenital Anomaly or | 0 | 0 | 0 | 0 | 0 | 0 | 0 | 0 | 0 | 0 | 0 | 0 | 0 | 0 | 0 | 0 |
| Birth Defect | | | | | | | | | | | | | | | | |
| Other Medically | 0 | 0 | 0 | 0 | 0 | 0 | 1 | 33.3 | 1 | 25.0 | 1 | 16.7 | 0 | 0 | 3 | 12.0 |
| Important Serious | | | | | | | | | | | | | | | | |
| Event | | | | | | | | | | | | | | | | |
| Subjects with | | | | | | | | | | | | | | | | |
| maximum CTC Grade | | | | | | | | | | | | | | | | |
| Grade 1 | 2 | 66.7 | 0 | 0 | 1 | 33.3 | 0 | 0 | 2 | 50.0 | 0 | 0 | 0 | 0 | 5 | 20.0 |
| Grade 2 | 1 | 33.3 | 1 | 33.3 | 2 | 66.7 | 2 | 66.7 | 1 | 25.0 | 2 | 33.3 | 0 | 0 | 9 | 36.0 |
| Grade 3 | 0 | 0 | 2 | 66.7 | 0 | 0 | 1 | 33.3 | 0 | 0 | 3 | 50.0 | 1 | 33.3 | 7 | 28.0 |
| Grade 4 | 0 | 0 | 0 | 0 | 0 | 0 | 0 | 0 | 1 | 25.0 | 1 | 16.7 | 2 | 66.7 | 4 | 16.0 |
| Grade 5 | 0 | 0 | 0 | 0 | 0 | 0 | 0 | 0 | 0 | 0 | 0 | 0 | 0 | 0 | 0 | 0 |

A subject may have serious AE(s) with multiple seriousness criteria.

Percentages are calculated using total number of subjects per treatment as the denominator.

MedDRA version used for reporting: 23.1

CTCAE v5.0 is used for reporting

### 3.1.3.3 Efficacy

Preliminary efficacy signals were observed with both administration schedules (arm A and arm B):
3 partial responses (PR) in Arm A (representing 10.3% of all tested patients): one in an MDM2-amplified biliary adenocarcinoma patient with 54% tumor shrinkage and two in MDM2-amplified well differentiated Liposarcoma (WDLPS) patients (see FIG. 3). One patient in Arm A even showed a duration of response of more than 2 years (see Table 4) and 19 patients in Arm A experienced a stable disease (representing 65.5% of all treated patients).
2 partial responses (PR) in Arm B (representing 8% of all tested patients): one in an MDM2-amplified well differentiated liposarcoma patient (see Table 5) and one in an MDM2-amplified pancreatic adenocarcinoma patient (PAC) with 41% tumor shrinkage (see FIG. 3). 15 patients in Arm B experienced a stable disease in arm B (representing 60% of all treated patients).

More details are outlined in Table 3. No clear difference was seen between the two administration schedules (arm A versus arm B).

TABLE 3
Comparison of efficacy between Arm A and Arm B of trial 1403-0001

| | Number of patients in | | Number of patients | | Number of patients | |
|---|---|---|---|---|---|---|
| | arm A (%) | | in arm B (%) | | in both arms (%) | |
| Total treated | 29 | (100) | 25 | (100) | 54 | (100) |
| Disease control | 22 | (75.9) | 17 | (68) | 39 | (72) |
| Partial response | 3 | (10.3) | 2 | (8) | 5 | (9.2) |
| Stable disease | 19 | (65.5) | 15 | (60) | 34 | (62.9) |
| Progressive disease | 6 | (20.7) | 5 | (20) | 11 | (20.3) |
| Notevaluable | 1 | (3.4) | 2 | (8) | 3 | (5.5) |
| Too early to assess | 0 | (0) | 1 | (4) | 1 | (1.8) |

Overall, 9% of the patients experienced a partial response (PR) and 63% showed a disease stabilization/stable disease (SD).

A treatment with a drug candidate (here the MDM2-antagonist of formula I) leading to a “PARTIAL RESPONSE (PR)” is defined by a ≥30% decrease in the sum of diameters of target lesions, taking as reference the sum of diameters of the target lesions prior to treatment (=baseline sum of diameters) according to the RECIST1.1 criteria (Eisenhauer et al, Eur. J. Canc. (2009), Vol. 45, pp. 228-247).

A treatment with a drug candidate (here the MDM2-antagonist of formula I) leading to a “PROGRESSIVE DISEASE (PD)” is defined by a ≥20% increase in the sum of diameters of target lesions, taking as reference the smallest sum of diameters of target lesions on study (including the baseline sum of diameters if that is the smallest sum of diameters of target lesions on study) according to the RECIST1.1 criteria (Eisenhauer et al, Eur. J. Canc. (2009), Vol. 45, pp. 228-247).

A treatment with a drug candidate (here the MDM2-antagonist of formula I) leading to a “STABLE DISEASE (SD)” is defined by neither sufficient shrinkage to qualify for PR nor sufficient increase to qualify for PD, taking as reference the smallest sum of diameters of the target lesions on study (including the baseline sum of diameters if that is the smallest sum of diameters on study) according to the RECIST1.1 criteria (Eisenhauer et al, Eur. J. Canc. (2009), Vol. 45, pp. 228-247).

When analyzing more closely the liposarcoma patients only and more specifically the eleven dedifferentiated liposarcoma (DDLPS) ones, the approximate median PFS was estimated around 10.8 months; a significant number of patients were progression free beyond 9 months as depicted in the swimmer plot of FIG. 1. These durable disease stabilizations starting at early dose levels (20 mg D1 q3w and 15/15 mg D1 D8 q4w) were noticeable as DDLPS patients experience one of the worst prognosis among the liposarcoma subtypes with a reported median Progression-free Survival (mPFS) between 2 and 4 months after SOC doxorubicin first line therapy (Italiano et, Ann. Oncol. (2012), Vol. 23, No. 6, pp. 1601-1607; Savina et al, BMC Medicine (2017), Vol. 15, pp. 78, Langmans et al, Onco. Res. Treat. (2019), Vol. 42, pp. 396-403).

These data are even more interesting when reviewing individual patient profiles. Indeed, as shown in the Table 4, the majority of the patients who experienced sustained durations of disease stabilization under administration of the MDM2-antagonist of formula I did actually progress under previous systemic therapies in 1^{st}, 2^{nd} and sometimes later lines of therapy.

Table 4 below shows the liposarcoma patients characteristics that were treated by the MDM2-antagonist of formula I in arm A.

TABLE 4
Characteristics of LPS patients treated in Arm A of trial 1403-0001

| | | | | | | | Best overall | |
|---|---|---|---|---|---|---|---|---|
| | | | | | | Dose | response | Current |
| | | | | | | (mg)(D 1q3w) | (days of | patient |
| | | MDM2 | TP53 | | Reason | of the | exposure to | status |
| | Tumor | Status | Status | Previous systemic therapy | for | MDM2i of | the MDM2i | (reason for |
| Patient ID | type* | ** | *** | (number of cycles) | discon. | formula I | of formula I | discon.) |
| 1392001001 | WD LPS | AMPL | WT | Investig cell therapy (13 cy) | PD | 20 | PR (778) | Discontinued |
| | | | | Doxoubicin (3 cy) | — | | | (PD) |
| | | | | Ezabenlimab (5 cy) | PD | | | |
| 1846002602 | DDLPS | AMPL | | Doxorubicin + Olar (3 cy) | PD | 45 | SD (709) | Discontinued |
| | | | | | | | | (PD) |
| 1840002004 | DDLPS | AMPL | | Doxorubicin + Olar (6 cy) | — | 45 | SD (353) | Discontinued |
| | | | | Riboci + Trametinib (24 cy) | — | | | (withdraw) |
| 1392001006 | DDLPS | AMPL | WT | Doxorubicin + Ifosfomide | PD | 50 | SD (107) | Discontinued |
| | | | | (2 cy) Eribulin (2 cy) | PD | | | |
| | | | | Trabectidin (2 cy) | PD | | | |
| 1840003006 | DDLPS | AMPL | WT | Doxorubicin + Ifosfomide (4 cy) | — | 50 | SD (85) | Discontinued |
| | | | | Palbociclib | PD | | | |
| | | | | Pembrolizumab (3 cy) | PD | | | |
| 1840003004 | DDLPS | AMPL | | No prior therapy reported | | 60 | SD (342) | Discontinued |
| | | | | | | | | (PD) |
| 1840003008 | DDLPS | AMPL | WT | Docetaxel + Gemcitabine (4 cy) | PD | 60 | SD (57) | Discontinued |
| | | | | Doxorubicin + Ifosfomide (2 cy) | PD | | | (PD) |
| 1392001011 | WDLPS | AMPL | | Doxorubicin (6 cy) | PD | 80 | PR (228) | ongoing |
| 1840003013 | WDLPS | AMPL | WT | Palbociclib | PD | 80 | SD (214) | ongoing |
| | | | | Pembrolizumab (13 cy) | PD | | | |
| | | | | Selinexor/placebo (6 cy) | PD | | | |

*DDLPS: dedifferentiated liposarcoma/WDLPS: well differentiated liposarcoma
** AMPL: MDM2 amplified
*** WT: TP53 wild type

As for arm A, the arm B patients characteristics are summarized in Table 5 showing that the majority of patients durably stabilized under the administration of the MDM2-antagonist of formula I while progressing under previous therapies in 1^{st}, 2^{nd} and later lines.

TABLE 5
Characteristics of LPS patients treated in Arm B of trial 1403-0001

| | | | | | | | Best overall | |
|---|---|---|---|---|---|---|---|---|
| | | | | | | Dose (mg) | response | Current |
| | | | | | | (D 1D 8q4w) | (days of | patient |
| | | MDM2 | TP53 | | | of the | exposure to | status |
| | Tumor | Status | Status | Previous systemic therapy | Reason for | MDM2i of | the MDM2i | (reason for |
| Patient ID | type* | ** | *** | (number of cycles) | discon. | formula I | of formula I) | discon.) |
| 1392001005 | DDLPS | AMPL | WT | Doxorubicin (2 cy) | PD | 15/15 | SD (491) | Discontinued |
| | | | | Ifosfomide (5 cy) | unknown | | | (PD) |
| | | | | Eribulin (3 cy) | PD | | | |
| 1840002005 | DDLPS | AMPL | WT | No prior therapy reported | | 20/20 | SD (542) | Discontinued |
| | | | | | | | | (withdraw) |
| 1392001007 | DDLPS | AMPL | WT | Doxorubucin (3 cy) | PD | 30/30 | SD (218) | Discontinued |
| | | | | Eribulin (16 cy) | unknown | | | (PD) |
| 1840003011 | WD LPS | AMPL | | Abemaciclib (23 cy) | — | 30/30 | SD (100) | Discontinued |
| | | | | | | | | (PD) |
| 1840003009 | DDLPS | AMPL | | Palbociclib (3 cy) | PD | 45/45 | SD (274) | Discontinued |
| | | | | | | | | (AE) |
| 1840003010 | DDLPS | AMPL | WT | Palbociclib | PD | 45/45 | SD (64d) | Discontinued |
| | | | | Doxorubicin/Olaratumab (2 cy) | PD | | | (PD) |
| | | | | Docetaxel + Gemcitabine (1 cy) | PD | | | |
| | | | | Eribulin (1 cy) | PD | | | |
| | | | | Pembrolizumab (1 cy) | PD | | | |
| | | | | Ifosfomide (1 cy) | PD | | | |
| | | | | Trabectidin (1 cy) | PD | | | |
| | | | | Dacarbazine (1 cy) | PD | | | |
| 1840003014 | WD LPS | AMPL | WT | Abemaciclib (1 cy) | — | 45/45 | SD (127) | Discontinued |
| | | | | Palbociclib (6 cy) | PD | | | (PD) |
| 1392001009 | WD LPS | AMPL | WT | Doxorubicin (2 cy) | PD | 60/60 | SD (105) | Discontinued |
| | | | | Eribulin (22 cy) | unknown | | | (PD) |
| 1840003012 | WD LPS | AMPL | WT | Palbociclib (3 cy) | PD | 60/60 | PR (221) | ongoing |

### 3.1.3.4 Bayesian Logistic Regression Model (BLRM) Analysis

Dose escalation in both Arm A and Arm B was determined and guided by a BLRM with overdose control (“R13-4803 Neuenschwander B, Branson M, Gsponer T. Critical aspects of the Bayesian approach to phase 1 cancer trials. Stat Med 2008. 27: 2420-2439”). Based on dose limiting toxicities (DLTs) reported during the maximum tolerated dose (MTD) evaluation period (Cycle 1), 60 mg in Arm A and 45 mg in Arm B were proposed as MTDs respectively since they were the highest doses that fulfilled the Escalation with Overdose Control (EWOC) criterion from the primary BLRM analysis (posterior probability of the DLT rate exceeding 33% is less than 25%) as well as the MTD criteria per protocol.

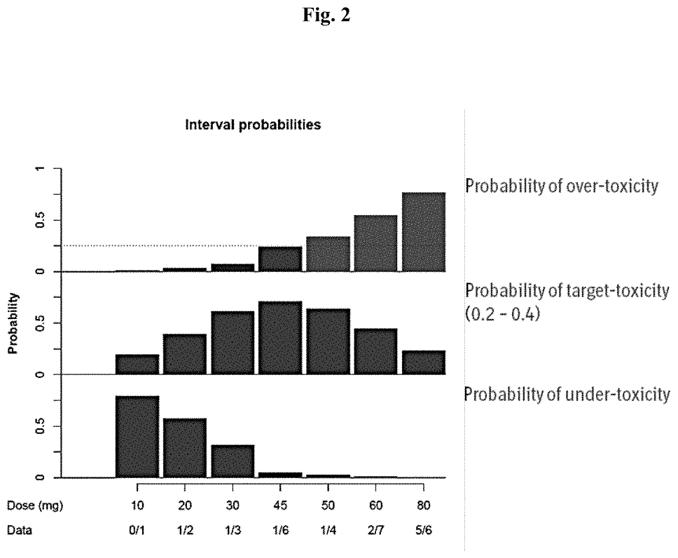

An additional BLRM analysis was conducted for Arm A considering additional events of interests during the entire treatment period, including hematologic DLTs in all cycles, Grade 4 hematologic AEs, hematologic AEs leading to dose delay/dose reduction/discontinuation, time for platelet count recovery (to over 100.000 10\hat{ }9/L) longer than 21 days, and time for neutrophil count recovery (to over 1.5 10\hat{ }9/L) longer than 21 days. As a result, 45 mg in Arm A was the highest dose level that fulfilled the EWOC criterion from this additional BLRM analysis (posterior probability of the event rate exceeding 40% is less than 25%) as outlined in the corresponding Table 6 and in the BLRM sensitivity analysis in FIG. 2. Consequently 45 mg was proposed as recommended Dose for expansion (RDE) for trial phase Ib. Besides 45 mg of the compound of formula I (which is the highest dose fulfilling the EWOC criterion) also 30 mg of the compound of formula I fulfills the EWOC criterion as the second highest dose.

TABLE 6
Number of AEs (throughout all cycles) considered for the
BLRM sensitivity analysis by dose level in all the patients
treated in Arm A of the 1403-0001 study

| | Dose of the | | |
|---|---|---|---|
| | MDM2i of | N of evaluable | N of evaluable patients |
| Arm | formula I | patients | with events of interests |
| A | 10 mg | 1 | 1 |
| A | 20 mg | 2 | 1 |
| A | 30 mg | 3 | 1 |
| A | 45 mg | 6 | 1 |
| A | 50 mg | 4 | 1 |
| A | 60 mg | 7 | 2 |
| A | 80 mg | 6 | 5 |

The outcome of the BLRM sensitivity analysis is shown in FIG. 2 and confirmed 45 mg D1 q3w as a safe dose for the patients even beyond cycle 1.

### 3.1.3.5 Conclusion

54 patients had been treated with the MDM2-antagonist of formula I in monotherapy in the Phase Ia dose escalation clinical trial (1403-0001), including 29 patients in Arm A (dosing regimen: D1 q3 weeks; dose range: 10-80 mg) and 25 patients in Arm B (D1 D8 q4 weeks; dose range: 5-60 mg). Based on safety, BLRM, efficacy and exploratory PK and PD analyses, it was decided that the MDM2-antagonist of formula I in monotherapy has Maximum Tolerated Doses (MTDs) at 60 mg for the dose regimen D1 q3w (Arm A) and 45 mg for the dose regiment D1 D8 q4w (Arm B). The Recommended Dose for Expansion (RDE) for the MDM2-antagonist of formula I in monotherapy for dose regimen D1 q3w was determined to be 45 mg.

3.1.4 Dose Finding for the MDM2-Antagonist of Formula I in Combination with Ezabenlimab as Supported by the Results of Trial 1403-0002

### 3.1.4.1 Safety

The dose escalation part of trial 1403-0002 assessing the initial triple combination of the MDM2-antagonist of formula I with ezabenlimab (anti-PD-1 mAb) and the anti-LAG 3 mAb anti-LAG3-1 reached the 45 mg dose level for the MDM2 antagonist of formula I without DLTs for the 2 patients enrolled at that dose level (see Table 7).

TABLE 7
Adverse event overall summary - 1403-0002

| | Comp I | | Comp. I | | Comp. I | | Comp. I | | Comp. I | |
|---|---|---|---|---|---|---|---|---|---|---|
| | 10 mg | | 20 mg | | 30 mg | | 45 mg | | Total | |
| | N | % | N | % | N | % | N | % | N | % |
| Number of subjects | 3 | 100 | 3 | 100 | 3 | 100 | 2 | 100 | 11 | 100 |
| Subjects with any AE | 3 | 100 | 3 | 100 | 3 | 100 | 2 | 100 | 11 | 100 |
| Patients with | 0 | 0 | 0 | 0 | 0 | 0 | 0 | 0 | 0 | 0 |
| Dose limiting toxicity | | | | | | | | | | |
| (cycle one) | | | | | | | | | | |
| Subjects with investigator | 3 | 100 | 3 | 100 | 3 | 100 | 2 | 100 | 11 | 100 |
| defined drug-related AEs | | | | | | | | | | |
| Subjects with AEs leading | 0 | 0 | 0 | 0 | 1 | 33.3 | 1 | 50.0 | 2 | 18.2 |
| to dose reduction of trial drug | | | | | | | | | | |
| Subjects with AEs leading | 0 | 0 | 0 | 0 | 0 | 0 | 0 | 0 | 0 | 0 |
| to Discontinuation of trial drug | | | | | | | | | | |
| Dose limiting toxicity | 0 | 0 | 0 | 0 | 0 | 0 | 0 | 0 | 0 | 0 |
| Other adverse event | 0 | 0 | 0 | 0 | 0 | 0 | 0 | 0 | 0 | 0 |
| Subjects with immune-related AEs | 0 | 0 | 1 | 33.3 | 0 | 0 | 0 | 0 | 1 | 9.1 |
| Subjects with infusion | 1 | 33.3 | 0 | 0 | 0 | 0 | 0 | 0 | 1 | 9.1 |
| related reactions | | | | | | | | | | |
| Subjects with serious AEs | 0 | 0 | 2 | 66.7 | 2 | 66.7 | 1 | 50.0 | 5 | 45.5 |
| Results in Death | 0 | 0 | 0 | 0 | 0 | 0 | 0 | 0 | 0 | 0 |
| Is Life Threatening | 0 | 0 | 0 | 0 | 0 | 0 | 0 | 0 | 0 | 0 |
| Persists of Signif. | 0 | 0 | 0 | 0 | 0 | 0 | 0 | 0 | 0 | 0 |
| Disability/Incapacity | | | | | | | | | | |
| Requires or Prolongs Hospitalization | 0 | 0 | 2 | 66.7 | 2 | 66.7 | 1 | 50.0 | 5 | 45.5 |
| Cogenital Anomaly or Birth Defect | 0 | 0 | 0 | 0 | 0 | 0 | 0 | 0 | 0 | 0 |
| Other Medically Important | 0 | 0 | 0 | 0 | 0 | 0 | 0 | 0 | 0 | 0 |
| Serious Event | | | | | | | | | | |
| Subjects with | | | | | | | | | | |
| maximum CTC Grade | | | | | | | | | | |
| Grade 1 | 1 | 33.3 | 0 | 0 | 0 | 0 | 0 | 0 | 1 | 9.1 |
| Grade 2 | 2 | 66.7 | 0 | 0 | 1 | 33.3 | 1 | 50.0 | 4 | 36.4 |
| Grade 3 | 0 | 0 | 3 | 100 | 1 | 33.3 | 0 | 0 | 4 | 36.4 |
| Grade 4 | 0 | 0 | 0 | 0 | 1 | 33.3 | 1 | 50.0 | 2 | 18.2 |
| Grade 5 | 0 | 0 | 0 | 0 | 0 | 0 | 0 | 0 | 0 | 0 |

Additionally, 15 patients had received the double combination of the MDM2-antagonist of formula I (10 patients in the 30 mg dose, 5 patients in the 45 mg dose) in combination with 240 mg ezabenlimab administered D1 q3w.
One patient had a DLT during cycle 1 (Grade 2): neutropenia (45 mg of the MDM2-antagonist of formula I)
Four DLTs were reported after cycle 1: Grade 3 anemia (30 mg of the MDM2-antagonist of formula I), Grade 2 thrombocytopenia (45 mg of the MDM2-antagonist of formula I), Grade 3 neutropenia (45 mg of the MDM2-antagonist of formula I), Grade 4 thrombocytopenia (45 mg of the MDM2-antagonist of formula I)

Grade ≥3 adverse events occurred in eight patients, most commonly anemia (6 patients), thrombocytopenia (4 patients) and lymphopenia (3 patients).

Consequently,—similarly as treatment with 30 mg or 45 mg of the MDM2-antagonist of formula I alone in the D1 q3w dose regimen—also the double combination of 30 mg or 45 mg of the MDM2-antagonist of formula I combined with 240 mg ezabenlimab (D1 q3w) shows a manageable safety profile.

### 3.1.4.2 Efficacy Summary for 1403-0002

Eleven patients have been dosed with the triple combination of the MDM2-antagonist of formula I, ezabenlimab (anti-PD-1 mAb) and the anti-LAG 3 mAb anti-LAG3-1 starting at 10 mg D1 q3w to 45 mg D1 q3w of the MDM2 antagonist of formula I until this triple combination was no longer pursued.

Preliminary signs of efficacy were observed with 2 Partial Responses (PRs) in an MDM2-amplified liposarcoma and an MDM2-amplified urothelial carcinoma patient. In addition, seven patients experienced a disease stabilization (representing 63.6% of all tested patients); Table 8 summarizes the patients' outcomes.

TABLE 8
Efficacy summary from trial 1403-0002

| | Number of | |
|---|---|---|
| | patients (%) | |
| Total treated | 11 | (100) |
| Disease control | 8 | (72.5) |
| Confirmed partial response | 1 | (9.1) |
| Unconfirmed partial response | 1 | (9.1) |
| Stable disease | 7 | (63.6) |
| Progressive disease | 2 | (18.2) |
| Not evaluable | 1 | (9.1) |

After the triple combination has been no longer pursued the 1403-0002 study has been resumed with the double combination of the MDM2-antagonist of formula I with ezabenlimab. As no overlapping toxicities between the MDM2-antagonist of formula I and ezabenlimab were expected and no dose escalation beyond 45 mg dose level was planned, 45 mg of the MDM2-antagonist of formula I was also the recommended dose for the combined therapy with 240 mg ezabenlimab q3w.

In summary, 26 patients (11 patients received the triple combination of the MDM2-antagonist of formula I with ezabenlimab and with the anti-LAG 3 antibody anti-LAG3-1; 15 patients received the double combination of the MDM2-antagonist of formula I with ezabenlimab only) had been enrolled in study 1403-0002 (10 patients in the 30 mg dose of the MDM2-antagonist of formula I, 5 patients in the 45 mg dose of the MDM2-antagonist of formula I, each in combination with 240 mg ezabenlimab administered D1 q3w).

Nine of the 15 patients who had received the double therapy (the MDM2-antagonist of formula I in combination with ezabenlimab) were evaluable for tumor response (see FIG. 5):
four patients had a confirmed partial response (PR): one patient who had received the 30 mg dose of the MDM2-antagonist of formula I in combination with 240 mg ezabenlimab (D1 q3w) and three patients who had received the 45 mg dose of the MDM2-antagonist of formula I in combination with 240 mg ezabenlimab (D1 q3w) (including two patients with biliary tract carcinoma, one with urothelial carcinoma, and one with myxoid liposarcoma)
one patient with adenocarcinoma had an unconfirmed PR (30 mg of the MDM2-inhibitor of formula I in combination with 240 mg ezabenlimab, D1 q3w)
Four patients in various tumor types had a stable disease (see FIG. 5).

Overall, both monotherapy and combination trials showed early signs of anti-tumor activity with PR in different tumor types—in majority MDM2-amplified tumor types, as shown in FIG. 4 and FIG. 5.

3.1.5 Disease Stabilizations were Also Seen in Patients with Non-MDM2-Amplified Cancers

As seen in trial 1403-0001 (both arms, see Table 3) overall 9% of the patients experienced a partial response (PR, tumor shrinkage of at least −30%) and overall 63% of the tested patients showed a disease stabilization (SD, tumor growth modification between +20% and −30%).

As outlined in FIG. 4, sustained disease stabilizations are seen mainly in MDM2-amplified tumors as expected and these were in majority liposarcoma patients. However, surprisingly also some cases of durable disease stabilizations could be observed in patients with p53 WT and “non-MDM2-amplified cancers” (with Copy Numbers (CN)=1, as determined e.g. by FISH (fluorescence in situ hybridization as described in Song et al, Appl Immunohistochem Mol Morphol, 2017, vol. 25(10), pp. 712-719):
one patient with “non-MDM2 amplified leiomyosarcoma” experienced a stable disease for more than 14 months by treatment with the MDM2-antagonist of formula I
one patient with “non-MDM2-amplified chondrosarcoma” experienced a stable disease for more than 10 months by treatment with the MDM2-antagonist of formula I
one patient with “non-MDM2-amplified melanoma” experienced a stable disease for more than 7 months by treatment with the MDM2-antagonist of formula I.
Consequently the clinical data show surprisingly that treatment with the MDM2-antagonist of formula I is also efficacious in p53 wildtype, but non-MDM2-amplified tumors.

### 3.1.6 Pharmacokinetics & Human Dose Prediction

Preliminary pharmacokinetic (PK) data were used as supportive information for the dose decision. The human therapeutic dose was predicted based on a minimum effective AUC₀₋₁₆₈ of 30,700 nM*h (QW), which was determined in a mouse SJSA-1 xenograft experiment. For this, the difference in plasma protein binding between human and mouse was taken into account, which translated to an effective AUC₀₋₁₆₈ of 11,350 nM*h for QW dosing in humans. The human therapeutic dose was predicted to be 64 mg (free acid) with QW dosing. The corresponding Cₘₐₓ was predicted with 680 nM.

Preliminary PK data were available from the first 45 treated patients in study 1403-0001. The exploratory PK analysis is based on planned blood sampling time-points and the results are subject to change because not all samples from these patients have been analyzed yet. Therefore, AUCs may be incomplete. In addition, information on the absorption, distribution, and elimination of the MDM2 antagonist of formula I are descriptive given the limited exploratory PK results available up to this point in time.

Available PK results indicate that plasma exposure of the MDM2 antagonist of formula I is increasing with increasing doses and the exposure (Cₘₐₓ and AUC_{0-inf}) is above predicted therapeutic exposures at dose levels of 15 mg or above (see Tables 9 and 10). In particular, Table 9 shows that patients that were dosed 30 mg or 45 mg of the MDM2-inhibitor of formula I once in three weeks (D1q3w) showed exposures (Cₘₐₓ and AUC_{0-inf}) that were clearly above the predicted therapeutic exposures. Consequently the preliminary PK results support the dose selection of 45 mg (and alternatively even of 30 mg) combined with the once in three weeks (D1 q3w) dose regimen. The tₘₐₓ of the MDM2 antagonist of formula I is generally between 4 and 6 hours after tablet intake. The half-life is in the range of 27.9 to 60.9 hours. Based on the preliminary PK data, the MDM2 antagonist of formula I has a low clearance and a very low volume of distribution in humans.

TABLE 9
Preliminary gMean (gCV [%]) PK parameters of the MDM2-antagonist
of formula I after administration of 10-80 mg D 1 q3w (Arm A)

| | | | | Cₘₐₓ | | C_{max, norm} | | AUC_{0-∞} |
|---|---|---|---|---|---|---|---|---|
| | | tₘₐₓ¹ | | [nmol/L] | | [nmol/L/mg] | | [nmol*h/L] |
| Dose | | [h] | | (gCV | | (gCV | | (gCV |
| [mg] | N | (range) | | [%]) | | [%]) | | [%]) |
| 10 | — | — | | — | | — | | — |
| 20 | 2 | 5.50 | (3.00-8.00) | 1160 | (7.91) | 58.2 | (7.91) | — |
| 30 | 3 | 4.00 | (4.00-6.00) | 2890 | (32.6) | 96.4 | (32.6) | 113000 (46.8) |
| 45 | 6 | 4.00 | (3.00-6.00) | 2710 | (62.4) | 60.2 | (62.4) | 137000 (53.6) |
| 50 | 4 | 5.50 | (2.00-24.0) | 3740 | (6.54) | 74.8 | (6.54) | 314000 (37.3) |
| 60 | 7 ² | 5.00 | (3.00-8.00) | 3270 | (26.6) | 54.5 | (26.6) | 163000 (33.4) |
| 80 | 2 | 3.50 | (2.00-5.00) | 3930 | (26.2) | 49.2 | (26.2) | 205000 (29.2) |

| | AUC_{0-∞, norm} | | | | | | | |
|---|---|---|---|---|---|---|---|---|
| | [nmol | % AUC_{tz-∞} | | t_{1/2} | | CL/F | | V_{z}/F |
| | *h/L/mg] | [%] | | [h] | | [mL/min] | | [L] |
| Dose | (gCV | (gCV | | (gCV | | (gCV | | (gCV |
| [mg] | [%]) | [%]) | | [%]) | | [%]) | | [%]) |
| 10 | — | — | | — | | — | | — |
| 20 | — | — | | — | | — | | — |
| 30 | 3760 (46.8) | 24.6 | (13.4) | 36.4 | (7.24) | 7.50 | (46.8) | 23.6 (46.2) |
| 45 | 3040 (53.6) | 29.2 | (45.8) | 43.7 | (40.7) | 9.28 | (53.6) | 35.1 (55.0) |
| 50 | 6280 (37.3) | 19.7 | (179) | 60.9 | (58.1) | 4.49 | (37.3) | 23.7 (32.3) |
| 60 | 2710 (33.4) | 3.71 | (47.5) | 34.9 | (11.7) | 10.4 | (33.4) | 31.4 (28.0) |
| 80 | 2570 (29.2) | 2.54 | (37.1) | 27.9 | (18.3) | 11.0 | (29.2) | 26.5 (49.4) |

¹Median and range; gMean, geometric mean; gCV, geometric coefficient of variation; — not calculated;
² N = 7 for Cₘₐₓ, C_{max, norm} and Tₘₐₓ; N = 6 for all other parameters of this dose group;

TABLE 10
Preliminary gMean (gCV [%]) PK parameters of the MDM2-antagonist of formula
I (gCV [%]) after administration of 5-60 mg D 1 D 8 q4w(ArmB)

| | | | | | | | | | | AUC_{0-∞, norm} | |
|---|---|---|---|---|---|---|---|---|---|---|---|
| | | | | Cₘₐₓ | | C_{max, norm} | | AUC_{0-∞} | | [nmol | |
| | | tₘₐₓ¹ | | [nmol/L] | | [nmol/L/mg] | | [nmol*h/L] | | *h/L/mg] | |
| Dose | | [h] | | (gCV | | (gCV | | (gCV | | (gCV | |
| [mg] | N | (range) | | [%]) | | [%]) | | [%]) | | [%]) | |
| Day 1 | 3 | 4.00 | (2.00-8.00) | 260 | (46.6) | 51.9 | (46.6) | 17600 | (105) | 3530 | (105) |
| 5 | | | | | | | | | | | |
| Day 8 | 3 | 4.00 | (3.00-8.00) | 336 | (12.2) | 67.2 | (12.2) | 19300 | (80.1) | 3860 | (80.1) |
| 5 | | | | | | | | | | | |
| Day 1 | 3 | 6.00 | (4.00-24.0) | 638 | (28.7) | 63.8 | (28.7) | 52300 | (70.9) | 5230 | (70.9) |
| 10 | | | | | | | | | | | |
| Day 8 | 3 | 5.00 | (5.00-8.00) | 714 | (53.3) | 71.4 | (53.3) | 48400 | (116) | 4840 | (116) |
| 10 | | | | | | | | | | | |
| Day 1 | 3 | 5.00 | (4.00-24.0) | 831 | (14.3) | 55.4 | (14.3) | 37800 | (36.4) | 2520 | (36.4) |
| 15 | | | | | | | | | | | |
| Day 8 | 3² | 4.00 | (3.00-24.0) | 776 | (22.9) | 51.7 | (22.9) | 25800 | (44.6) | 1720 | (44.6) |
| 15 | | | | | | | | | | | |
| Day 1 | 3 | 4.00 | (3.00-8.00) | 1370 | (13.8) | 68.4 | (13.8) | 88900 | (38.8) | 4440 | (38.8) |
| 20 | | | | | | | | | | | |
| Day 8 | 3 | 3.00 | (2.00-4.00) | 1220 | (11.9) | 60.9 | (11.9) | 61100 | (50.2) | 3050 | (50.2) |
| 20 | | | | | | | | | | | |
| Day 1 | 4 | 4.50 | (4.00-8.00) | 1440 | (37.8) | 48.0 | (37.8) | 79900 | (25.9) | 2660 | (25.9) |
| 30 | | | | | | | | | | | |
| Day 8 | 3 | 4.00 | (4.00-6.00) | 1330 | (18.8) | 44.3 | (18.8) | 64800 | (8.95) | 2160 | (8.95) |
| 30 | | | | | | | | | | | |
| Day 1 | 2 | 4.00 | (4.00-4.00) | 2330 | (42.9) | 51.7 | (42.9) | 96900 | (27.4) | 2150 | (27.4) |
| 45 | | | | | | | | | | | |
| Day 8 | 0 | — | | — | | — | | — | | — | |
| 45 | | | | | | | | | | | |
| Day 1 | 2 | 6.00 | (4.00-8.00) | 1960 | (137) | 32.7 | (137) | 87700 | (299) | 1460 | (299) |
| 60 | | | | | | | | | | | |

| | | | | | | | | | |
|---|---|---|---|---|---|---|---|---|---|
| Day 8 | 2 | 16.00 | (8.00-24.00) | 1890 | (6.75) | 31.5 | (6.75) | — | — |
| 60 | | | | | | | | | |

| | % AUC_{tz-∞} | | t_{1/2} | | CL/F | | V_{z}/F | |
|---|---|---|---|---|---|---|---|---|
| | [%] | | [h] | | [mL/min] | | [L] | |
| Dose | (gCV | | (gCV | | (gCV | | (gCV | |
| [mg] | [%]) | | [%]) | | [%]) | | [%]) | |
| Day 1 | 10.2 | (145) | 55.3 | (49.6) | 7.99 | (105) | 38.2 | (40.9) |
| 5 | | | | | | | | |
| Day 8 | 30.2 | (62.3) | 46.2 | (52.3) | 7.31 | (80.1) | 29.2 | (21.5) |
| 5 | | | | | | | | |
| Day 1 | 10.8 | (68.6) | 52.4 | (26.9) | 5.39 | (70.9) | 24.5 | (43.6) |
| 10 | | | | | | | | |
| Day 8 | 31.6 | (67.2) | 45.9 | (58.7) | 5.82 | (116) | 23.1 | (40.0) |
| 10 | | | | | | | | |
| Day 1 | 2.89 | (123) | 33.4 | (28.9) | 11.2 | (36.4) | 32.3 | (21.8) |
| 15 | | | | | | | | |
| Day 8 | 14.8 | (8.69) | 26.3 | (8.37) | 16.4 | (44.6) | 37.3 | (35.3) |
| 15 | | | | | | | | |
| Day 1 | 8.63 | (116) | 50.5 | (39.7) | 6.34 | (38.8) | 27.7 | (18.5) |
| 20 | | | | | | | | |
| Day 8 | 25.9 | (65.3) | 38.2 | (42.1) | 9.22 | (50.2) | 30.5 | (7.77) |
| 20 | | | | | | | | |
| Day 1 | 6.72 | (160) | 36.2 | (17.0) | 10.6 | (25.9) | 33.1 | (31.8) |
| 30 | | | | | | | | |
| Day 8 | 18.9 | (11.8) | 39.5 | (7.31) | 13.0 | (8.95) | 44.6 | (8.47) |
| 30 | | | | | | | | |
| Day 1 | 4.72 | (337) | 30.0 | (13.3) | 13.1 | (27.4) | 34.0 | (41.8) |
| 45 | | | | | | | | |
| Day 8 | — | | — | | — | | — | |
| 45 | | | | | | | | |
| Day 1 | 7.08 | (3.84) | 34.2 | (46.9) | 19.3 | (299) | 57.0 | (146) |
| 60 | | | | | | | | |

| | | | | |
|---|---|---|---|---|
| Day 8 | — | — | — | — |
| 60 | | | | |

¹Median and range; gMean, geometric mean; gCV, geometric coefficient of variation; — not calculated;
²N = 3 for Cₘₐₓ, C_{max, norm} and Tₘₐₓ; N = 2 for all other parameters of this dose group

### 3.1.7 First Line Therapies

The term ‘first-line treatment or first-line therapy’ usually implies for oncology the administration of systemic anti-cancer therapy to patients with unresectable/inoperable advanced or/and metastatic cancer as an initial treatment with palliative intent (that is, non-curative/life-extending intent) (Saini et al, Brit. J. Canc. (2021), Vol. 125 pp. 155-163; Judson et al, Lancet Oncology (2014), Vol. 15, pp. 415-423).

In the trial 1403-0001 one DDLPS patient on arm A (see Table 4) and another DDLPS patient on arm B (see Table 5), both with no prior history of systemic therapy, experienced long disease stabilization and stayed successfully on treatment for 342 and 542 days, respectively. These durable disease stabilizations in DDLPS patients with no prior systemic therapy were remarkable, as DDLPS patients experience one of the worst prognosis among the liposarcoma subtypes with a reported median Progress-free Survival (mPFS) between 2 and 4 months even after Standard of Care (SOC) first line therapy with doxorubicin. This shows that treatment with the MDM2-antagonist of formula I is also suitable for “first line treatment” or “primary treatment” of dedifferentiated liposarcoma patients and seems to show even better therapeutic efficacy in dedifferentiated liposarcoma patients than the well-established SOC treatment with doxorubicin.

What is claimed is:

1. A method of treating cancer, comprising orally administering to a patient in need thereof an oral pharmaceutical composition comprising the MDM2-antagonist of formula I 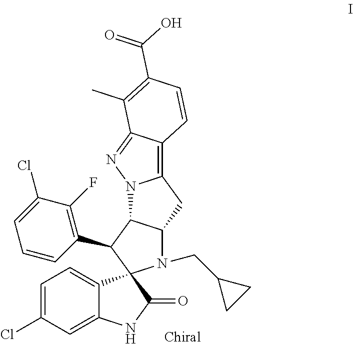 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
in a dose range of 30 mg to 45 mg, wherein the method comprises administering said oral pharmaceutical composition is administered in a treatment cycle of once every three weeks (D1 q3w), wherein optionally this treatment cycle of once every three weeks (D1 q3w) may be repeated as many times as considered beneficial for the patient from a medical point of view.

2. The method according to claim 1, wherein the cancer that is treated is a p53 wildtype form of cancer.

3. The method according to claim 1, wherein the cancer that is treated is a soft tissue sarcoma.

4. The method according to claim 1, wherein the cancer that is treated is liposarcoma.

5. The method according to claim 1, wherein the cancer that is treated is well differentiated liposarcoma (WDLPS) or dedifferentiated liposarcoma (DDLPS).

6-7. (canceled)

8. The method according to claim 1, wherein the MDM2-antagonist of formula I is present in a 45 mg dose or a 30 mg dose.

9-18. (canceled)

19. A method of first line systemic treatment (primary treatment) of dedifferentiated liposarcoma (DDLPS), comprising administering to a patient in need thereof an effective amount of an MDM2-antagonist of formula I

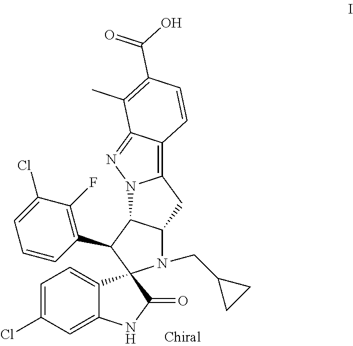 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]

20. The method of claim 19, wherein said MDM2 antagonist is administered to the patient in need thereof in a treatment cycle of once every three weeks (D1 q3w), wherein optionally this treatment cycle may be repeated as many times as considered beneficial for the patient from a medical point of view.

21. The method according to claim 19, wherein the MDM2-antagonist is administered to the patient in each treatment cycle in a dose range of 30 mg to 45 mg.

22. The method of claim 19, wherein the MDM2-antagonist is administered to the patient in each treatment cycle in a dose of 45 mg or 30 mg.

23-28. (canceled)

29. A method of treating cancer, comprising orally administering to a patient in need thereof an MDM2-antagonist of formula I 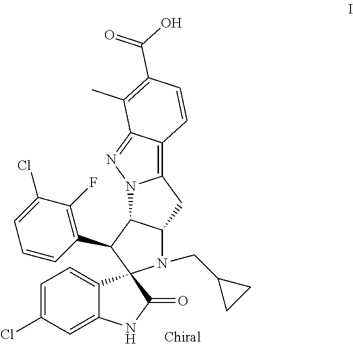 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
wherein the MDM2-antagonist of formula I is administered in combination with ezabenlimab and wherein
both the MDM2-antagonist and ezabenlimab are administered to the patient in need thereof in a treatment cycle of once every three weeks (D1 q3w);
the MDM2-antagonist is administered in a dose of 45 mg;
ezabenlimab is administered in a dose of 240 mg; and
optionally, this treatment cycle may be repeated as many times as considered beneficial for the patient from a medical point of view.

30. The method according to claim 29, wherein the cancer is a p53 wildtype form of cancer.

31. The method according to claim 29, wherein the cancer is a soft tissue sarcoma.

32. The method according to claim 29, wherein the cancer is liposarcoma.

33. The method according to claim 29, wherein the cancer is a liposarcoma selected from the group consisting of a dedifferentiated liposarcoma (DDLPS) and a well differentiated liposarcoma (WDLPS).

34-35. (canceled)

36. The method according to claim 29, wherein the treatment of cancer is a first line treatment.

37. The method according to claim 29, wherein the 45 mg of the MDM2-antagonist is administered orally and wherein the 240 mg ezabenlimab is administered intravenously.

38. A method of treating cancer, comprising orally administering to a patient in need thereof an MDM2-antagonist of formula I 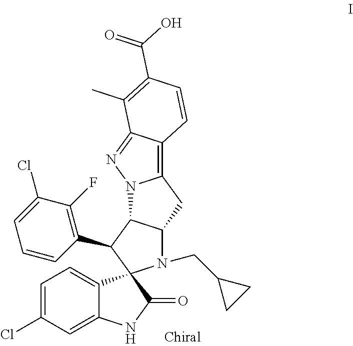 [SMILES: Cc1c(C(=O)O)ccc2c3n(nc12)[C@@H]1[C@H](C3)N(CC2CC2)[C@@]2(C(=O)Nc3cc(Cl)ccc32)[C@H]1c1cccc(Cl)c1F]
wherein said cancer is a p53-wildtype and non-MDM2-amplified form of cancer.

39. The method according to claim 38, wherein the p53 wildtype and non-MDM2-amplified form of cancer is selected from the group consisting of leiomyosarcoma, chondrosarcoma and melanoma.

40-41. (canceled)
